(19) United States
(12) Patent Application Publication
(10) Pub. No.: US 2006/0165626 A1
(43) Pub. Date: Jul. 27, 2006

(54) Cosmetic composition comprising at least one ester of alkoxylated alcohol and at least one film-forming polymer

(76) Inventors: Audrey Ricard, Paris (FR); Caroline Lebre, Thiais (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/321,872

(22) Filed: Dec. 30, 2005

Related U.S. Application Data
(60) Provisional application No. 60/642,969, filed on Jan. 12, 2005.

(30) Foreign Application Priority Data
Dec. 30, 2004 (FR) 0453268

(51) Int. Cl. A61K 8/86; A61K 8/36

(52) U.S. Cl. 424/70.11; 424/70.31

(57) ABSTRACT

Disclosed herein is a composition comprising at least one ester of an alkoxylated alcohol and of a carboxylic acid and at least one solid noncrystalline film-forming polymer. The ester of an alkoxylated alcohol may, for example, be Octyidodecyl PPG-3 Myristyl Ether Dimer Dilinoleate. In one embodiment, this composition may be used as a product for caring for and/or making up keratinous substances, for example, the skin, lips, and/or superficial body growths.

This application claims benefit of U.S. Provisional Application No. 60/642,969, filed Jan. 12, 2005, the contents of which are incorporated herein by reference. This application also claims benefit of priority under 35 U.S.C. § 119 to French Patent Application No. 04 53268, filed Dec. 30, 2004, the contents of which are also incorporated herein by reference.

Disclosed herein is a cosmetic composition, for example, a cosmetic composition for making up and/or caring for the skin of the human face or body, of the scalp, of the lips, and/or of the superficial body growths, such as the hair, eyelashes, eyebrows, and nails.

In one embodiment, the composition of the present disclosure may be a product for making up the lips, body, and/or superficial body growths which may additionally possess care properties. The composition of the present disclosure may be a product chosen from lipsticks, lip glosses, face powders, eyeshadows, body painting products, mascaras, eyeliners, nail varnishes, products for the artificial tanning of the skin, and products for coloring and/or caring for the hair.

In one embodiment, the cosmetic composition according to the present disclosure may exhibit a very satisfactory hold of the color while being lustrous and comfortable.

Hold is a desirable property for users of cosmetic products. Lipsticks with satisfactory hold are generally sticks comprising ester oils of low molecular weight or silicone oils. In order to enhance the hold of these products, previous formulation concepts have been based on the combination of a “lustrous” phase and of a “color and comfort” phase rendered compatible by a volatile solvent. When the product is applied to the lips, the volatile material evaporates and then a phenomenon of segregation occurs. However, this concept may be limited in improving the hold and the comfort because the range of compatibility of the phases remains restricted and the level of isododecane cannot be increased at the expense of the comfort and hardness of the sticks.

Formulations have been provided comprising acrylate/acrylic copolymers dispersed in a volatile solvent, such as isododecane. These polymers form a film on the lips after the evaporation of the solvent. Nevertheless, very high levels of volatile materials are usually necessary in these formulations in order to ensure the hold of the cosmetic composition on keratinous substances, at the expense of the comfort of the layer deposited on the keratinous substances.

The present inventors have discovered that, in one embodiment, an alkoxylated ester in combination with certain film-forming polymers makes it possible to obtain formulations with improved lustre and with a comfort equivalent to that of a conventional formulation, while improving the hold of the color in comparison with known formulations.

Some alkoxylated esters have been used in cosmetic compositions.

For example, U.S. patent application Publication No. 2002/0192249 discloses cosmetic compositions comprising an ester of a monocarboxylic acid comprising from 4 to 24 carbon atoms and of an alcohol comprising a polypropoxyl group and an alkyl chain comprising from 2 to 24 carbon atoms. In addition to the alkoxylated ester, the preparations may comprise mineral oil or liquid paraffin. This document also discloses an anhydrous composition comprising this ester and a film-forming agent, and a composition comprising PPG-3 Myristyl Ether Neoheptanoate. More specifically, the application describes lipstick compositions comprising this propoxylated ester in combination with hydrogenated polyisobutene; cream foundation compositions as an emulsion comprising this ester; and sunscreen compositions as an emulsion comprising hexyl laurate, octyl palmitate, and cetyl palmitate in combination with this ester.

U.S. Pat. No. 5,693,316 discloses cosmetic compositions comprising an alkoxylated fatty ester obtained from a dicarboxylic acid having from 2 to 22 carbon atoms, for example, maleic acid, and from a stoichiometric excess of at least one polyalkoxylated fatty alcohol comprising an alkyl chain comprising from 14 to 22 carbon atoms and a polyalkoxyl group. The preparations may comprise mineral oil or liquid paraffin as second emollient. This patent also discloses an anhydrous composition comprising this ester and a film-forming agent. The alkoxylated ester may be Di-PPG-3 Myristyl Maleate.

U.S. Pat. No. 6,476,254 discloses cosmetic compositions comprising an ester of a dicarboxylic acid comprising from 4 to 12 carbon atoms and of a polyalkoxylated fatty alcohol, the nonalkoxylated part of which comprises from 8 to 36 carbon atoms. The ester may be Di-PPG-3 Myristyl Adipate. The composition may be anhydrous and may comprise mineral oil or liquid paraffin.

International Application Publication No. WO 2003/013439 describes an ester of a C₃-C₂₁ dicarboxylic acid or of an aliphatic C₄-C₂₂, for example, C₃ to C₉, tricarboxylic acid and of a polyalkoxylated fatty alcohol comprising a C₆-C₃₀, for example, C₁₈-C₂₂, alkyl radical. This publication discloses a cosmetic composition which may comprise petrolatum, mineral oil, esters of aliphatic carboxylic acids and of aliphatic alcohols comprising from 18 to 40 carbon atoms, a film-forming agent, or a fatty alcohol, such as cetyl alcohol.

U.S. Pat. Nos. 5,302,377, 5,455,025, and 5,597,555 disclose cosmetic compositions comprising an alkoxylated fatty ester of a tricarboxylic acid, for example, citric acid, with a stoichiometric excess of at least one polyalkoxylated fatty alcohol having emollient properties for topical preparations. The preparations may comprise a mineral oil as second emollient. This patent also discloses a combination of this ester with a film-forming agent. The ester may be, for example, Tri-PPG-3 Myristyl Citrate.

International Application Publication No. WO 2004/052076 discloses cosmetic compositions comprising mixed esters of polyalkoxylated alcohols and of monohydric alcohols with polycarboxylic acids, for example, dicarboxylic acids. These compositions may comprise a second emollient agent, such as mineral oil or petrolatum. The mixed esters disclosed may be formulated in combination with a film-forming compound.

Disclosed herein, in one embodiment, is a cosmetic composition comprising at least one ester of an alkoxylated alcohol and of a carboxylic acid and at least one noncrystalline film-forming polymer which is solid at ambient temperature.

As used herein, the term “solid at ambient temperature” is understood to mean a polymer which does not flow under its own weight. A polymer in the form of a powder is solid within the meaning of the present disclosure. As regards a viscous or very viscous polymer, the ability to flow under its own weight may, for example, be evaluated by placing 20 g of the polymer on a support and by plotting the outline of the deposited material immediately afterwards with a black felt-tip pen. The sample is thus left for approximately 2 hours at a temperature adjusted to 25° C. At the end of this period of time, no flow outside the region of deposition is observed with the naked eye.

Also disclosed herein is a cosmetic composition comprising at least one ester of an alkoxylated alcohol and of a carboxylic acid and at least one noncrystalline film-forming polymer chosen from vinyl, for example, acrylic and ethylenic polymers and copolymers; urethane polymers and copolymers; polyester polymers and copolymers; polyamide polymers and copolymers, for example, silicone polyamides; polyurea polymers and copolymers; cellulose polymers and copolymers, such as nitrocellulose; and silicone polymers and copolymers.

In one embodiment, the composition may be capable of forming a deposited layer having a hold index of greater than or equal to 30%.

In another embodiment, when the the film-forming polymer is in the composition in a sufficient amount, the composition may be capable of forming a deposited layer having a hold index of greater than or equal to 30%.

Hold

In at least one embodiment, the composition may be capable of forming a deposited layer having a hold index of greater than or equal to 30%, for example, greater than or equal to 40%, greater than or equal to 45%, or greater than or equal to 50%.

The hold index of the deposited layer obtained with the composition according to the present disclosure is determined according to the measurement protocol described below.

A support (40 mm×70 mm rectangle) composed of an acrylic coating (hypoallergenic acrylic adhesive on a polyethylene film, sold under the name Blenderm, ref. FH5000-55113, by 3M Health Care) adhesively bonded to a layer of polyethylene foam is prepared which is adhesive on the face opposite that to which the plaster is attached (layer of foam sold under the name RE40X70EP3 by Joint Technique Lyonnais Ind).

The color L*₀ₐ*_{0b}*₀ of the support, the side of the acrylic coating face, is measured using a Minolta CR 300 calorimeter.

The support thus prepared is preheated on a heating plate maintained at a temperature of 40° C. in order for the surface of the support to be maintained at a temperature of 33° C.±1° C.

While leaving the support on the heating plate, the composition is applied over the entire nonadhesive surface of support (that is to say, over the surface of the acrylic coating) by spreading it using a brush, in order to obtain a deposited layer of the composition approximately 15 μm thick, and then drying is allowed to take place for 10 minutes.

After drying, the color L*a*b* of the film thus obtained is measured.

The difference in color ΔE1 between the color of the film with respect to the color of the bare support is then determined by the following relationship:
ΔE1={square root over (L*−Lₒ*)²+(a*−aₒ*)²+(b*−bₒ*)²)}

The support is subsequently adhesively bonded by its adhesive face (adhesive face of the layer of foam) to an anvil with a diameter of 20 mm provided with a thread. A test specimen of the support/deposited layer combination is subsequently cut out using a hollow punch with a diameter of 18 mm. The anvil is subsequently screwed over a press (Statif Manuel Imada SV-2 from Someco) equipped with a tensile testing device (Imada DPS-20 from Someco).

A strip with a width of 33 mm and a length of 29.7 cm is drawn on a blank photocopier paper with a grammage of 80 g/m², a first line is plotted 2 cm from the edge of the sheet and then a second line is plotted 5 cm from the edge of the sheet, the first and second lines thus delimiting a box on the strip; then a first mark and a second mark situated in the strip are positioned respectively at the points 8 cm and 16 cm from the second line. 20 μl of water are placed on the first mark and 10 μl of refined sunflower oil (sold by Lesieur) are placed on the second mark.

The blank paper is placed over the bed of the press and then the test specimen, placed over the box of the strip of paper, is pressed to a pressure of approximately 300 g/cm² exerted for 30 seconds. The press is then raised up and the test specimen is again placed immediately after the second line (thus beside the box), a pressure of approximately 300 g/cm² is again applied and the paper is moved, rectilinearly from the contact produced, with a speed of 1 cm/s over the entire length of the strip, so that the test specimen passes over the deposits of water and of oil.

After removing the test specimen, a portion of the deposited layer has transferred onto the paper. The color L*′a*′b*′ of the deposited layer remaining on the test specimen is then measured.

The difference in color ΔE2 between the color of the deposited layer remaining on the test specimen with respect to the color of the bare support is then determined by the following relationship:
ΔE2=\sqrt{(L*′−Lₒ*)²+(a*′−aₒ*)²+(b*′−bₒ*)²}

The hold index of the composition, expressed as a percentage, is equal to the ratio 100×ΔE2/ΔE1.

The measurement is carried out on 6 supports in succession and the hold index corresponds to the mean of the 6 measurements obtained with the 6 supports.

Also disclosed herein is a cosmetic method for conferring properties of lustre, of hold, and/or of comfort on a film of cosmetic composition which comprises introducing, into said composition, at least one ester of an alkoxylated alcohol and of a carboxylic acid and at least one film-forming polymer as defined above.

Further disclosed herein is a method for caring for and/or making up keratinous substances comprising applying, to the keratinous substances, a composition comprising at least one ester of an alkoxylated alcohol and of a carboxylic acid and at least one film-forming polymer as defined above, the composition being capable of forming a deposited layer having a hold index of greater than or equal to 30%.

As used herein, the term “alkoxylated alcohol” is understood to mean a hydrocarbon compound comprising at least one —OH functional group and at least one group of formula (I)

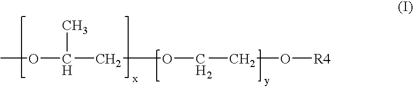

in which

x and y, which may be identical or different, are integers ranging from 0 to 40 inclusive, wherein the sum of x and y ranges from 1 to 80 inclusive, and

R₄ is chosen from substituted or unsubstituted, saturated or unsaturated, and aliphatic or aromatic hydrocarbon units comprising from 1 to 36 carbon atoms, for example, from 4 to 36 carbon atoms. In at least one embodiment, the alkoxylated alcohol may comprise one hydroxyl group.

The alkoxylated alcohol may be a polyalkoxylated alcohol, for example, a group of formula (I) in which x and y are independently integers ranging from 0 to 40 inclusive, wherein the sum of x and y is between 2 and 80 inclusive. In on embodiment, x and y may be integers from 0 to 30 inclusive, wherein the sum of x and y may be between 2 and 30 inclusive.

Formula (I) illustrates diagrammatically all the ethoxy units in a first group and all the propoxy units in another group. However, these units can be placed in any order, randomly, in blocks, or in the form of alternating units. Purely by way of illustration, the ethoxy units (E) and the propoxy units (P) of the alkoxylated alcohol may be positioned in an arrangement chosen from EEEP, EEPE, EPEE, PEEE, EEEPEPPPE, PEPPPEEEEPE, and similar arrangements.

Alkoxylated Ester

The composition according to the present disclosure may comprise at least one ester of an alkoxylated alcohol and of a carboxylic acid, referred to hereinafter as an alkoxylated ester, which may be chosen from:

esters obtained by reaction of a monocarboxylic acid with an alkoxylated alcohol,

polyesters obtained by reaction of a polycarboxylic acid with a stoichiometric excess of at least one alkoxylated alcohol with respect to the number of acid functional groups of said acid,

polyesters, one ester functional group of which is obtained by reaction of an acid functional group of a polycarboxylic acid with an alkoxylated alcohol,

polyesters comprising at least one ester functional group obtained by reaction of an acid functional group of a polycarboxylic acid with an alkoxylated alcohol and at least one ester functional group obtained by reaction of another acid functional group of the said polycarboxylic acid with a fatty alcohol, and

mixtures thereof.

Alkoxylated Ester of a Monocarboxylic Acid

The alkoxylated ester may be chosen from esters of a monocarboxylic acid and of alkoxylated fatty alcohols, such as polyalkoxylated fatty alcohols. For example, the alkoxylated ester may be chosen from esters formed by the reaction of an aliphatic or aromatic monocarboxylic acid with a stoichiometric excess of polyalkoxylated fatty alcohol, for example, a polypropoxylated alcohol.

The alkoxylated ester of a monocarboxylic acid may be chosen from polypropoxylated monoesters of formula (II):

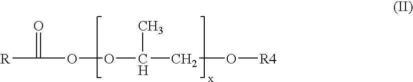

in which

x is an integer ranging from 2 to 40 inclusive, for example, from 3 to 30, or from 3 to 10,

R₄ is chosen from substituted or unsubstituted and saturated or unsaturated aliphatic hydrocarbon units comprising from 1 to 36 carbon atoms, for example, from 3 to 24 carbon atoms, or from 4 to 24 carbon atoms, and

RCOO is chosen from aliphatic and aromatic monocarboxylic acids RCOOH.

RCOO may be chosen from:

residues of monocarboxylic acids, for example, acids of formula (R₂R₃R₄C)COO in which R₂, R₃, and R₄ are independently chosen from methyl, ethyl, propyl, and isopropyl groups; and

residues of an aromatic acids comprising a benzene ring optionally substituted by a group chosen from —OH, —NH₂, methyl, and ethyl.

The aliphatic monocarboxylic acids suitable for the preparation of the alkoxylated ester may comprise from 4 to 24 carbon atoms, for example, from 4 to 18 carbon atoms. Examples of aliphatic monocarboxylic acids include, but are not limited to, 2-ethylhexanoic acid, caproic acid, neopentanoic acid, isostearic acid, neoheptanoic acid, and oleic acid.

Non-limiting examples of aromatic monocarboxylic acids include benzoic acid and p-aminobenzoic acid.

The alkoxylated alcohols used to prepare the alkoxylated esters may be saturated or unsaturated, substituted or unsubstituted, and aliphatic or aromatic and may be straight-chain or branched-chain. They may comprise from 6 to 24 carbon atoms, for example, from 12 to 14 carbon atoms.

As used herein, the term “fatty alcohol” is understood to mean an aliphatic alcohol comprising at least three carbon atoms. In at least one embodiment, the fatty alcohol may comprise carbon, hydrogen, and oxygen atoms. The fatty alcohol may be saturated or may comprise at least one carbon-carbon double bond.

A fatty alcohol may, for example, be an alcohol obtained by hydrolysis of vegetable fats, animal fats, vegetable oils, or animal oils.

The esters of a monocarboxylic acid and of a polypropoxylated fatty alcohol may, for example, be chosen from PPG-3 Myristyl Ether Neoheptanoate, sold under the reference Trivasperse, PPG-4 Butyloctyl Ether Ethylhexanoate, and their mixtures.

These esters may be prepared, for example, according to the disclosure of U.S. patent application Publication No. 2002/0192249, which is incorporated herein by reference.

Alkoxylated Mixed Esters

The alkoxylated ester may be chosen from mixed esters of an alkoxylated alcohol and of a monohydric alcohol with polycarboxylic acids, for example, dicarboxylic acids. For example, the alkoxylated ester may be chosen from mixed esters of a polyalkoxylated fatty alcohol and of a monohydric fatty alcohol with dicarboxylic fatty acids.

As used herein, the term “mixed ester” is understood to mean an ester obtained by reaction of a polycarboxylic acid with at least two different alcohols.

The mixed ester of an alkoxylated alcohol can, for example, be chosen from compounds of formula (III):

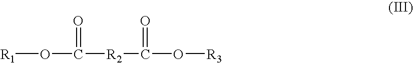

in which

R₁ is chosen from groups of formula (IIIa):

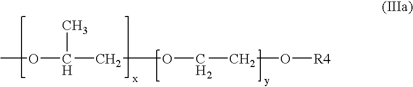

in which:

R₄ is chosen from saturated or unsaturated and substituted or unsubstituted aliphatic units comprising from 4 to 24 carbon atoms;

x is an integer ranging from 3 to 30;

y is an integer ranging from 3 to 30;

R₂ is chosen from saturated or unsaturated and substituted or unsubstituted aliphatic units comprising from 4 to 40 carbon atoms; and

R₃ is chosen from saturated or unsaturated, straight-chain or branched-chain aliphatic units comprising from 4 to 32 carbon atoms, for example, from 12 to 24 carbon atoms.

Examples of compounds corresponding to formula (III) include, but are not limted to:

Octyldodecyl PPG-3 Myristyl Ether Dilinoleate, sold under the reference Liquiwax PolyEFA by Arch Chemical, of formula (IV):

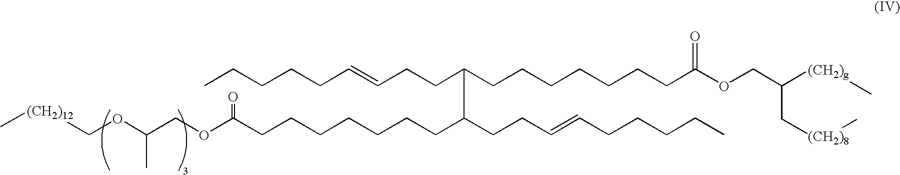

Stearyl PPG-3 Myristyl Ether Dilinoleate, sold under the reference Liquiwax PolyIPL by Arch Chemical, and

Isostearyl PPG-4 Butyloctyl Ether Dilinoleate.

These mixed esters may be produced by the reaction of alkoxylated fatty alcohols and of monohydric fatty alcohols with dicarboxylic fatty acids.

In at least one embodiment, the alkoxylated fatty alcohols may be propoxylated fatty alcohols having a carbon chain length ranging from 4 to 24 carbon atoms and a degree of propoxylation ranging from 3 to 30, for example, from 3 to 15 propylene oxide units. The propoxylated fatty alcohols may be chosen, for instance, from myristyl alcohol and butyloctanol.

The dicarboxylic acid may comprise at least two carboxyl groups per molecule. They may, for example, be represented by formula (V):
HOOC—(CH₂)ₙ—COOH (V)

in which n is an integer ranging from 1 to 16, for example, from 3 to 16.

Non-limiting examples of suitable dicarboxylic acids include malonic acid, succinic acid, glutaric acid, adipic acid, pimelic acid, suberic acid, azelaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenediacarboxylic acid, 1,14-tetradecamethylenedicarboxylic acid, 1,15-penta-decamethylenedicarboxylic acid, 1,16-hexadecamethylenedicarboxylic acid, and mixtures thereof.

In on embodiment, the dicarboxylic acid may also be a dimer diacid. As used herein, a dimer diacid denotes a diacid obtained by an intermolecular polymerization, for instance, dimerization, reaction of at least one unsaturated monocarboxylic acid.

Dimer diacids derive, for example, from the dimerization of an unsaturated fatty acid, such as an unsaturated C₈ to C₃₄ fatty acid, for instance an unsaturated C₁₂ to C₂₂ fatty acid, an unsaturated C₁₆ to C₂₀ fatty acid, or an unsaturated C₁₈ fatty acid.

Examples of suitable unsaturated fatty acids include, but are not limited to, undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, arachidonic acid, and mixtures thereof.

According to one embodiment, the dimer diacid may be that from which the dimer diol to be esterified also derives. For example, it may be the dimer diacid obtained by dimerization of linoleic acid, optionally followed by hydrogenation of the carbon-carbon double bonds. The dimer diacid may be in the saturated form, i.e., may not comprise any carbon-carbon double bond. According to another embodiment, the possible carbon-carbon double bonds of the dimer diacid may be all or partially hydrogenated, after the esterification reaction of the dimer diacid with the dimer diol.

According to another embodiment of the present disclosure, the dimer diacid may be the commercial product comprising a dicarboxylic acid having 36 carbon atoms. This product may also comprise a trimeric acid and a monomeric acid, in proportions which depend on the degree of purity of the product. Conventionally, products with a content of dimer diacid of greater than 70% and products with a content of dimer diacid which has been adjusted to at least 90% are available commercially.

Dimer diacids, for example, dilinoleic diacids, the stability of which with regard to oxidation has been improved by hydrogenation of the double bonds remaining after the dimerization reaction, are also available commercially.

Any dimer diacid currently commercially available may be used in the present disclosure.

The monohydric fatty alcohols may have a carbon chain length ranging from 12 to 24. Non-limiting examples of monohydric fatty alcohols include octyldodecanol and isostearyl alcohol.

Examples of the preparation of the esters described above are given, for instance, in International Application Publication No. WO 2004/052076, which is incorporated herein by reference in its entirety.

Alkoxylated Polyesters

The alkoxylated ester may be obtained by esterification of a polycarboxylic acid with at least two alkoxylated alcohols, which may be identical or different, so as to form an ester.

The ester may, for example, be chosen from esters of formula (VI):

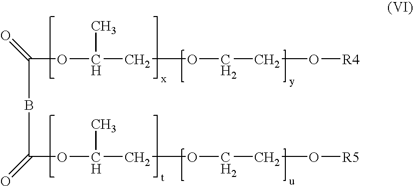

in which:

—OOC—B—COO— is chosen from residues of a saturated or unsaturated and substituted or unsubstituted dicarboxylic acid as described above, comprising, for example, from 2 to 40 carbon atoms, wherein B is a linking group containing up to 38 carbon atoms or a bond,

x and y, which may be identical or different, are integers ranging from 0 to 40 inclusive, wherein the sum of x and y ranges from 1 to 80 inclusive, for example, from 20 to 80 inclusive,

t and u, which may be identical or different are integers ranging from 0 to 40 inclusive, wherein the sum of t and u ranges from 1 to 80 inclusive, for example, from 2 to 80 inclusive, and

R₄ and R₅ are, independently of one another, chosen from substituted or unsubstituted, saturated or unsaturated, and aliphatic or aromatic hydrocarbon units comprising from 4 to 36 carbon atoms.

According to one embodiment, R₄ and R₅ may be identical or different and may comprise, for example, from 10 to 22 carbon atoms and may be substituted or unsubstituted and saturated or unsaturated.

According to another embodiment, y may range from 1 to 40 and x may range from 0 to 30, provided that, if x is equal to 0, y is equal to at least 2, and with the additional proviso that y is greater than x.

According to a further embodiment, u may range from 1 to 40 and t may range from 0 to 30, provided that, if t is equal to 0, u is equal to at least 2, and with the additional proviso that u is greater than t.

The dicarboxylic acid may be aliphatic and may comprise from 2 to 36 carbon atoms, for example, from 8 to 36 carbon atoms. In at least one embodiment, the aliphatic dicarboxylic acids may comprise from 3 to 8 carbon atoms. Suitable examples of aliphatic dicarboxylic acids include, but are not limited to, adipic acid, sebacic acid, malonic acid, succinic acid, and maleic acid.

The dicarboxylic acid may be aromatic and may comprise from 8 to 12 carbon atoms. A non-limiting example of a suitable aromatic dicarboxylic acid is phthalic acid, for example, 1,2-Phthalic acid, which has the lowest melting point of the isomers of phthalic acid.

In one embodiment, x and y may each be less than or equal to 15, the total of x and y not exceeding 25. In another embodiment, u and t may each be less than or equal to 15, the total of u and t not exceeding 25.

In a further embodiment, y and u may be greater than or equal to 1 and x and t are greater than or equal to 0. In yet another embodiment, the number of ethoxy units may be greater than the number of propoxy units.

The alkoxylated esters may be prepared, for example, according to the disclosure of International Application Publication No. WO 00/19972, which is incorporated herein by reference in its entirety.

The diesters of dicarboxylic acids may be chosen, for example, from unsubstituted, saturated, aliphatic groups comprising, for example, from 14 to 18 carbon atoms, or from 14 to 16 carbon atoms. In at least one embodiment, the diester of a dicarboxylic acid may be the myristyl fatty group comprising 14 carbon atoms.

In one embodiment, when R₄ and R₅ are myristyl groups, y and u may be equal to zero and x and t may be independently chosen from integers ranging from 2 to 40 inclusive. A non-limiting example of a commercial product is the product sold under the name Cromollient DP3A, in which, in the preceding formula, R₄ and R₅ are myristyl groups, —OOC—B—COO— is an adipate, y and u are equal to 0, and x and t are equal to 3.

The at least one alkoxylated ester may be present in the composition in an amount ranging from 1 to 99%, for example, from 2 to 60% by weight, from 5 to 40% by weight, or from 10 to 35% by weight, with respect to the total weight of the composition.

Film-Forming Polymer

As used herein, the term “film-forming polymer” may be understood as meaning a polymer capable of forming, by itself alone or in the presence of an additional agent which is able to form a film, for instance, a continuous film, on a support such as a keratinous substance, and/or a cohesive film, for example, a film, the cohesion and mechanical properties of which are such that the said film can be isolated from the support.

According to one embodiment, the at least one film-forming polymer is not crystalline. For example, the polymer may be insoluble in the oil(s) of the composition at its softening temperature, unlike a wax, even of polymeric origin, which is soluble in the oil(s) of the composition at its melting point.

Examples of film-forming polymers which may be used in the composition of the present disclosure include, but are not limited to, synthetic polymers, for instance, radical type and polycondensate type synthetic polymers; polymers of natural origin; and mixtures thereof.

Non-limiting examples of film-forming polymers include acrylic polymers; polyurethanes; polyesters; polyamides; polyureas; cellulose polymers, such as nitrocellulose; silicone polymers; and silicone polyamides.

The at least one film-forming polymer may be present in the composition in an amount ranging from 0.01% to 50%, with respect to the total weight of the composition, for example, from 1% to 30%, or from 5 to 25% by weight.

In at least one embodiment, the at least one film-forming polymer may be an organic polymer chosen from:

fat-soluble film-forming polymers,

fat-dispersible film-forming polymers, for example, polymers in the form of nonaqueous dispersions of polymer particles, such as dispersions in silicone oils and dispersions in hydrocarbon oils,

aqueous dispersions of particles of film-forming polymers, often referred to as “latexes”; in this case, the composition may comprise an aqueous phase, and

water-soluble film-forming polymers; in this case, the composition may comprise an aqueous phase.

Dispersion of Particles of a Grafted Ethylenic Polymer in a Liquid Fatty Phase

The composition according to the present disclosure may comprise, as film-forming agent, a dispersion of particles of a grafted ethylenic polymer in a liquid fatty phase.

As used herein, the term “‘ethylenic’ polymer” is understood to mean a polymer obtained by polymerization of monomers comprising an ethylenic unsaturation.

The dispersion of particles of a grafted ethylenic polymer may be devoid of stabilizing polymer separate from the said grafted polymer, such as those disclosed in European Patent No. 0 749 747 and described below. The particles of grafted ethylenic polymer are therefore not stabilized at the surface by such additional stabilizing polymers and are thus dispersed in the liquid fatty phase in the absence of additional stabilizer at the surface of the particles.

As used herein, the term “‘grafted’ polymer” is understood to mean a polymer having a backbone comprising at least one pendent side chain or one side chain situated at the chain end. In one embodiment, the backbone of the grafted polymer may comprise one pendent side chain.

According to at least one embodiment, the grafted ethylenic polymer may comprise an ethylenic backbone which is insoluble in the liquid fatty phase and side chains covalently bonded to said ethylenic backbone which are soluble in the liquid fatty phase.

The grafted ethylenic polymer may be a noncrosslinked polymer. For example, the polymer may be obtained by polymerization of monomers comprising only one polymerizable group.

According to one embodiment of the present disclosure, the grafted ethylenic polymer may be a grafted acrylic polymer.

The grafted ethylenic polymer may be obtained by radical polymerization, in an organic polymerization medium:

of at least one ethylenic monomer, for example, of at least one acrylic monomer and optionally of at least one additional nonacrylic vinyl monomer, in order to form the said insoluble backbone; and

of at least one macromonomer comprising an end group which can be polymerized to form the side chains, said macromonomer having a weight-average molecular mass of greater than or equal to 200, wherein the content of polymerized macromonomer represents from 0.05 to 20% by weight of the polymer.

The liquid fatty phase may comprise the organic medium for polymerization of the grafted ethylenic polymer.

The dispersing liquid organic medium, corresponding to the medium in which the grafted polymer is supplied, may be identical to the polymerization medium. However, the polymerization medium may be replaced all or in part by another liquid organic medium. This other liquid organic medium may be added, after polymerization, to the polymerization medium. The latter may be subsequently evaporated, all or in part.

The liquid fatty phase may comprise liquid organic compounds other than those present in the dispersing medium. These other compounds may be chosen so that the grafted polymer remains in the dispersed state in the liquid fatty phase.

The dispersing liquid organic medium may be present in the liquid fatty phase of the composition according to the present disclosure due to the introduction of the grafted polymer dispersion into the composition.

In at least one embodiment, the liquid fatty phase may predominantly comprise at least one liquid organic compound (or oil) as defined below.

For instance, the liquid fatty phase may be a nonaqueous liquid organic phase which is immiscible with water at ambient temperature (25° C.).

As used herein, the term “liquid organic compound” is understood to mean a nonaqueous compound which is in the liquid state at ambient temperature (25° C.) and which thus flows under its own weight.

As used herein the term “silicone compound” is understood to mean a compound comprising at least one silicon atom.

The composition according to the present disclosure may further comprise a volatile oil. The volatile oil may be chosen from silicone oils and nonsilicone oils. It may be chosen, for example, from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, isododecane, isodecane, isohexadecane, and mixtures thereof.

The volatile oil may be present in the composition in an amount ranging from 1% to 70% by weight, with respect to the total weight of the composition, for example, from 5% to 50% by weight, or from 10% to 35% by weight.

The liquid fatty phase may comprise a nonvolatile oil as described below. The nonvolatile oil may be present in the composition in an amount ranging from 1% to 80% by weight, with respect to the total weight of the composition, for example, from 5% to 60% by weight, or from 10% to 50% by weight.

Non-limiting examples of liquid organic compounds or oils which may be present in the dispersing liquid organic medium, include:

liquid organic compounds, for example, nonsilicone and silicone compounds, having an overall solubility diameter according to the Hansen solubility space of less than or equal to 18 (MPa)^{1/2}, for example, of less than or equal to 17 (MPa)^{1/2};

monoalcohols having an overall solubility parameter according to the Hansen solubility space of less than or equal to 20 (MPa)^{1/2}; and

mixtures thereof.

The overall solubility parameter δ according to the Hansen solubility space is defined, for example, in the article “Solubility parameter values” by Eric A. Grulke in Chapter VII of Polymer Handbook, 3rd edition, pp. 519-559, by the relationship:
δ=(δ_{D}²+δ_{P}²+δ_{H}²)^{1/2}

in which:

d_{D} characterizes the London dispersion forces resulting from the formation of dipoles induced during molecular impacts,

d_{P} characterizes the forces of Debye interactions between permanent dipoles, and

d_{H} characterizes the forces of specific interactions (hydrogen bond, acid/base, donor/acceptor type, and the like).

The definition of the solvents in the solubility space according to Hansen is described, for example, in the paper by C. M. Hansen, “The three-dimensional solubility parameters”, J. Paint Technol., 39, 105 (1967).

Examples of suitable liquid organic compounds include, but are not limited to, nonsilicone and silicone compounds, having an overall solubility parameter according to the Hansen solubility space of less than or equal to 18 (MPa)^{1/2}, liquid fatty substances, for instance, oils, which may be chosen from optionally branched, carbon, hydrocarbon, fluorinated, silicone, natural, synthetic oils, and mixtures thereof.

Non-limiting examples of oils include vegetable oils formed by esters of fatty acids and of polyols, for example, triglycerides, such as sunflower oil, sesame oil, and rapeseed oil, and vegetable oils formed by esters derived from long-chain acids or alcohols (i.e., esters comprising from 6 to 20 carbon atoms), for instance, esters of formula RCOOR′ in which R is chosen from residues of higher fatty acids comprising from 7 to 19 carbon atoms and R′ is chosen from hydrocarbon chains comprising from 3 to 20 carbon atoms, such as palmitates, adipates, and benzoates, for example, diisopropyl adipate.

Additional examples of suitable liquid organic compounds include optionally volatile, linear, branched, and/or cyclic alkanes and liquid paraffins, liquid petrolatum, hydrogenated polyisobutylene, isododecane, “Isopars” (volatile isoparaffins), esters, ethers, and ketones.

Further examples of liquid organic compounds include silicone oils, such as polydimethylsiloxanes and polymethylphenylsiloxanes, optionally substituted by optionally fluorinated aliphatic and/or aromatic groups or by functional groups, such as hydroxyl, thiol, and/or amine groups, and volatile silicone oils, for example, cyclic oils.

Suitable liquid organic compounds may also include, for example, optionally branched, volatile, and/or nonvolatile silicone oils.

As used herein, the term “volatile oil” is understood to mean an oil capable of evaporating from the skin or lips in less than one hour, having, for example, a vapour pressure, at ambient temperature and atmospheric pressure, ranging from 10⁻³ to 300 mmHg (0.13 Pa to 40 000 Pa).

The volatile silicone oils which may be used in the composition of the present disclosure may be chosen from linear or cyclic silicones comprising from 2 to 7 silicon atoms, these silicones optionally comprising alkyl or alkoxy groups comprising from 1 to 10 carbon atoms. Such silicones may include, for example, octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, and mixtures thereof.

Non-limiting examples of nonvolatile silicone oils include nonvolatile polydialkylsiloxanes, such as nonvolatile polydimethylsiloxanes (PDMS); polydimethylsiloxanes comprising at least one pendent group chosen from alkyl, alkoxy, and phenyl groups, (i.e., at least one group chosen from alkyl, alkoxy, and phenyl groups at the end of the silicone chain), which groups comprise from 2 to 24 carbon atoms; phenylated silicones, such as phenyl trimethicones, phenyl dimethicones, phenyl(trimethylsiloxy)diphenylsiloxanes, diphenyl dimethicones, diphenyl(methyldiphenyl)trisiloxanes; and polymethylphenylsiloxanes; polysiloxanes modified with at least one group chosen from fatty acids (for instance, C₈-C₂₀ fatty acids), fatty alcohols (for example, C₈-C₂₀ fatty alcohols),and polyoxyalkylenes (for instance, polyoxyethylene and/or polyoxypropylene); aminated polysiloxanes; polysiloxanes comprising hydroxyl groups; fluorinated polysiloxanes comprising a pendent fluorinated group (i.e., a fluorinated group at the end of the silicone chain) cinorusubg from 1 to 12 carbon atoms, all or part of the hydrogens of which are substituted by fluorine atoms; and mixtures thereof.

Suitable nonsilicone liquid organic compounds may be chosen from those having an overall solubility parameter according to the Hansen solubility space of less than or equal to 18 (MPa)^{1/2}, for example:

linear, branched, or cyclic esters comprising at least 6 carbon atoms, for example, from 6 to 30 carbon atoms;

ethers comprising at least 6 carbon atoms, for example, from 6 to 30 carbon atoms; and

ketones comprising at least 6 carbon atoms, for example, from 6 to 30 carbon atoms.

As used herein, the term “liquid monoalcohols having an overall solubility parameter according to the Hansen solubility space of less than or equal to 20 (MPa)^{1/2}” is understood to mean liquid aliphatic fatty monoalcohols comprising from 6 to 30 carbon atoms, the hydrocarbon chain not comprising a substituent group. Suitable monoalcohols according to the present disclosure include oleyl alcohol, hexyldecanol, decanol, octyldodecanol, and linoleyl alcohol.

According to one embodiment of the present disclosure, the liquid fatty phase may be a nonsilicone liquid fatty phase.

As used herein, the term “nonsilicone liquid fatty phase” is understood to mean a fatty phase comprising at least one nonsilicone liquid organic compound or oil such as those mentioned above, said nonsilicone compounds being present predominantly in the liquid fatty phase, i.e., present in an amount greater than or equal to 50% by weight, for instance, from 50 to 100% by weight, from 60% to 100% by weight (for example, from 60 to 99% by weight), or from 65% to 100% by weight (for example, from 65 to 95% by weight), with respect to the total weight of the liquid fatty phase.

The nonsilicone liquid organic compounds may be chosen, for example, from:

nonsilicone liquid organic compounds having an overall solubility parameter according to the Hansen solubility space of less than or equal to 18 (MPa)^{1/2};

monoalcohols having an overall solubility parameter according to the Hansen solubility space of less than or equal to 20 (MPa)^{1/2}; and

mixtures thereof.

The nonsilicone liquid fatty phase may thus optionally comprise silicone liquid organic compounds or oils, such as those mentioned above, which may be present in an amount of less than 50% by weight, for example, ranging from 0.1 to 40% by weight, from 1 to 35% by weight, or from 5 to 30% by weight, with respect to the total weight of the liquid fatty phase.

According to one embodiment of the present disclosure, the nonsilicone liquid fatty phase may not comprise silicone liquid organic compounds or oils.

When the liquid fatty phase is a nonsilicone liquid fatty phase, the macromonomers present in the grafted polymer may be chosen from carbon macromonomers as described below.

For example, when the liquid fatty phase is a nonsilicone liquid fatty phase, the grafted polymer present in the composition may be chosen from nonsilicone grafted polymers.

As used herein, the term “nonsilicone grafted polymer” is understood to mean a grafted polymer comprising predominantly a carbon macromonomer and optionally comprising 7% or less by weight, for example, 5% or less by weight of silicone macromonomer, or is devoid of silicone macromonomer.

According to another embodiment of the present disclosure, the liquid fatty phase may be a silicone liquid fatty phase.

As used herein, the term “silicone liquid fatty phase” is understood to mean a fatty phase comprising at least one silicone liquid organic compound or silicone oil such as those described above, said silicone compounds being present predominantly in the liquid fatty phase, i.e., present in an amount greater than or equal to 50% by weight, for instance, from 50 to 100% by weight, from 60% to 100% by weight (for example, from 60 to 99% by weight), or from 65% to 100% by weight (for instance, from 65 to 95% by weight), with respect to the total weight of the liquid fatty phase.

The at least one silicone liquid organic compound may, for example, be chosen from silicone liquid organic compounds having an overall solubility parameter according to the Hansen solubility space of less than or equal to 18 (MPa)^{1/2}.

The silicone liquid fatty phase may thus optionally comprise nonsilicone liquid organic compounds or oils, such as described above, which may be present in an amount of less than 50% by weight, for example, ranging from 0.1 to 40% by weight, from 1 to 35% by weight, or from 5 to 30% by weight, with respect to the total weight of the liquid fatty phase.

According to one embodiment of the present disclosure, the silicone liquid fatty phase may not comprise nonsilicone liquid organic compounds.

When the liquid fatty phase is a silicone liquid fatty phase, the macromonomers present in the grafted polymer may be chosen from silicone macromonomers as described below.

For example, when the liquid fatty phase is a silicone liquid fatty phase, the grafted polymer present in the composition may be chosen from silicone grafted polymers.

As used herein, the term “silicone grafted polymer” is understood to mean a grafted polymer comprising predominantly a silicone macromonomer and optionally comprising 7% or less by weight, for example, 5% or less by weight of carbon macromonomer, or is devoid of carbon macromonomer.

The choice of the monomers constituting the backbone of the polymer, of the macromonomers, the molecular weight of the polymer, and the proportion of the monomers and of the macromonomers may be made according to the dispersing liquid organic medium so as to advantageously obtain a dispersion of particles of grafted polymers, for example, a stable dispersion, it being possible for this choice to be made by a person skilled in the art.

As used herein, the term “stable dispersion” is understood to mean a dispersion which is not capable of forming a solid deposit or of showing liquid/solid phase separation, for instance, after centrifuging, for example, at 4000 revolutions/minute for 15 minutes.

The grafted ethylenic polymer forming the dispersed particles may thus comprise a backbone which is insoluble in said dispersing medium and a part which is soluble in said dispersing medium.

In at least one embodiment of the present disclosure, the grafted ethylenic polymer may be a random polymer.

As used herein, the term “grafted ethylenic polymer” is understood to mean a polymer capable of being obtained by radical polymerization of at least one ethylenic monomer with at least one macromonomer in an organic polymerization medium.

According to the present disclosure, the term “grafted acrylic polymer” is understood to mean a polymer capable of being obtained by radical polymerization of at least one acrylic monomer and optionally of at least one additional nonacrylic vinyl monomer with at least one macromonomer in an organic polymerization medium.

The at least one acrylic monomer may be present in an amount ranging from 50 to 100% by weight, for example, from 55 to 100% by weight (for instance, from 55 to 95% by weight), or from 60 to 100% by weight (for instance, from 60 to 90% by weight), based on the total weight of the acrylic monomers+optional nonacrylic vinyl monomers mixture.

The acrylic monomers may be chosen from monomers, the homopolymer of which is insoluble in the dispersing medium under consideration, i.e., the homopolymer is in solid (or undissolved) form in said dispersing medium at ambient temperature (20° C.) and at a concentration of greater than or equal to 5% by weight.

As used herein, the term “macromonomer comprising an end group which can be polymerized” is understood to mean any polymer comprising, on one of its ends, an end group which may be polymerized and which is capable of reacting during the polymerization reaction with the acrylic monomers and optionally the additional nonacrylic vinyl monomers constituting the backbone. The macromonomer thus may make it possible to form the side chains of the grafted acrylic polymer. The end group which can be polymerized may be chosen from groups comprising ethylenic unsaturation capable of polymerizing by the radical route with the monomers constituting the backbone.

As used herein, the term “carbon macromonomer” is understood to mean a nonsilicone macromonomer, for example, an oligomeric macromonomer obtained by polymerization of at least one nonsilicone monomer comprising ethylenic unsaturation and mainly by polymerization of acrylic and/or nonacrylic vinyl monomers.

As used herein, the term “silicone macromonomer” is understood to mean an organopolysiloxane macromonomer, for example, a polydimethylsiloxane macromonomer.

In at least one embodiment, the macromonomers may be chosen from macromonomers, the homopolymer of which is soluble in the dispersing medium under consideration, i.e., capable of completely dissolving in said dispersing medium at ambient temperature (20° C.) and at a concentration of greater than or equal to 5% by weight.

Thus, the grafted acrylic polymer may comprises a backbone (or main chain) comprising a series of acrylic units resulting from the polymerization, for example, of at least one acrylic monomer, and side chains (or grafts) resulting from the reaction of the at least one macromonomer, said side chains being covalently bonded to said main chain.

The backbone (or main chain) may be insoluble in the dispersing medium under consideration, whereas the side chains (or grafts) may be soluble in said dispersing medium.

As used herein, the term “acrylic monomers” is understood to mean monomers chosen from (meth)acrylic acid, esters of (meth)acrylic acid (also known as (meth)acrylates), and amides of (meth)acrylic acid (also known as (meth)acrylamides).

Examples of acrylic monomers suitable for being employed to form the insoluble backbone of the polymer, alone or as a mixture, include compounds (i),(ii), and/or (iii), as described below, and their salts:

(i) (meth)acrylates of formula (VII):

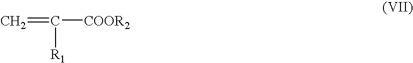

in which:

R₁ is chosen from hydrogen and methyl;

R₂ is chosen from:

linear or branched alkyl groups comprising from 1 to 6 carbon atoms, it being possible for said groups to comprise, in their chains, at least one heteroatom chosen from O, N, and S; and/or it being possible for said groups to comprise at least one substituent chosen from —OH, halogen atoms (F, Cl, Br, and 1), and —NR′R″, in which R′ and R″, which may be identical or different, are chosen from linear or branched C₁-C₄ alkyls; and/or it being possible for said groups to be substituted by at least one polyoxyalkylene group, for example, with a C₂-C₄ alkylene, such as polyoxyethylene and/or polyoxypropylene, said polyoxyalkylene group comprising the repetition of 5 to 30 oxyalkylene units;

cyclic alkyl groups comprising from 3 to 6 carbon atoms, it being possible for said groups to comprise, in their chain, at least one heteroatom chosen from O, N, and S and/or it being possible for the said groups to comprise at least one substituent chosen from OH and halogen atoms (F, Cl, Br, and I).

Examples of R₂ include, but are not limited to, methyl, ethyl, propyl, butyl, isobutyl, methoxyethyl, ethoxyethyl, methoxypolyoxyethylene 350 OE, trifluoroethyl, 2-hydroxyethyl, 2-hydroxypropyl, dimethylaminoethyl, diethylaminoethyl, and dimethylaminopropyl groups.

(ii) (meth)acrylamides of formula (VIII):

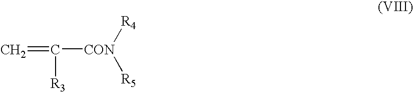

in which:

R₃ is chosen from hydrogen and methyl;

R₄ and R₅, which may be identical or different, are chosen from hydrogen and linear or branched alkyl groups comprising from 1 to 6 carbon atoms which may comprise at least one substituent chosen from —OH, halogen atoms (F, Cl, Br, and 1), and —NR′R″, in which R′ and R″, which may be identical or different, are chosen from linear or branched C₁-C₄ alkyls; or alternatively,

R₄ is hydrogen and R₅ is a 1,1-dimethyl-3-oxobutyl group.

Examples of R₄ and R₅ include, but are not limited to, n-butyl, t-butyl, n-propyl, dimethylaminoethyl, diethylaminoethyl, and dimethylaminopropyl.

(iii) (meth)acrylic monomers comprising at least one functional group chosen from carboxylic, phosphoric, and sulphonic acid functional groups, such as acrylic acid, methacrylic acid, and acrylamidopropanesulphonic acid.

Non-limiting examples of acrylic monomers include methyl, ethyl, propyl, butyl, and isobutyl (meth)acrylates; methoxyethyl and ethoxyethyl (meth)acrylates; trifluoroethyl methacrylate; dimethylaminoethyl methacrylate; diethylaminoethyl methacrylate; 2-hydroxypropyl methacrylate; 2-hydroxyethyl methacrylate, 2-hydroxypropyl acrylate; 2-hydroxyethyl acrylate; dimethylaminopropylmethacrylamide; and their salts; and mixtures thereof.

In at least one embodiment, the at least one acrylic monomer may be chosen from methyl acrylate, methoxyethyl acrylate, methyl methacrylate, 2-hydroxyethyl methacrylate, acrylic acid, dimethylaminoethyl methacrylate, and mixtures thereof.

Examples of nonacrylic vinyl monomers include, but are not limited to:

vinyl esters of formula: R₆—COO—CH═CH₂

in which R₆ is chosen from linear or branched alkyl groups comprising from 1 to 6 carbon atoms; cyclic alkyl groups comprising from 3 to 6 carbon atoms; and/or aromatic groups, for example of the benzene, anthracene, and naphthalene types;

nonacrylic vinyl monomers comprising at least one functional group chosen from carboxylic, phosphoric, and sulphonic acid functional groups, such as crotonic acid, maleic anhydride, itaconic acid, fumaric acid, maleic acid, styrenesulphonic acid, vinylbenzoic acid, vinylphosphoric acid, and their salts;

nonacrylic vinyl monomers comprising at least one tertiary amine functional group, such as 2-vinylpyridine and 4-vinylpyridine; and

mixtures thereof.

In one embodiment of the present disclosure, the at least one acrylic monomer present in the grafted polymer may comprise (meth)acrylic acid and at least one monomer chosen from the (meth)acrylates and the (meth)acrylamides described above at points (i) and (ii). In another embodiment, the at least one acrylic monomer may comprise (meth)acrylic acid and at least one monomer chosen from C₁-C₃ alkyl (meth)acrylates. (Meth)acrylic acid may be present in an amount equal to at least 5% by weight, with respect to the total weight of the polymer, for instance, ranging from 5% to 80% by weight, (for example, at least 10% by weight), from 10% by weight to 70% by weight, (for example, at least 15% by weight), or from 15% to 60% by weight.

Non-limiting examples of suitable salts of compounds (i), (ii), and (iii), include, those obtained by neutralization of the acidic groups using inorganic bases, such as sodium hydroxide, potassium hydroxide,and ammonium hydroxide, and/or organic bases of alkanolamine type, such as monoethanolamine, diethanolamine, triethanolamine, and 2-methyl-2-amino-1-propanol.

Additional examples of salts of compounds (i), (ii), and (iii) include, but are not limited to, those formed by neutralization of tertiary amine units, for example using inorganic or organic acid. Suitable inorganic acids may be chosen, for example, from sulphuric acid, hydrochloric acid, hydrobromic acid, hydriodic acid, phosphoric acid, and boric acid. Non-limiting examples of organic acids include acids comprising at least one group chosen from carboxyl, sulpho, and phosphono groups. The organic acids may be chosen from linear, branched, or cyclic aliphatic acids and aromatic acids and may additionally comprise at least one heteroatom chosen from O and N, for example, in the form of hydroxyl groups. Examples of such organic acids include, but are not limited to, acetic acid, propionic acid, terephthalic acid, citric acid, and tartaric acid.

According to one embodiment of the present disclosure, the grafted ethylenic polymer may not comprise additional nonacrylic vinyl monomers as described above. In this embodiment, the insoluble backbone of the grafted ethylenic polymer comprises solely acrylic monomers as described above.

It is to be understood that these unpolymerized acrylic monomers may be soluble in the dispersing medium under consideration but the polymer formed with these monomers is insoluble in the dispersing medium.

According to one embodiment of the present disclosure, the grafted ethylenic polymer may be capable of being obtained by radical polymerization, in an organic polymerization medium:

of at least one main acrylic monomer chosen from C₁-C₃ alkyl (meth)acrylates, alone or as a mixture, and optionally of at least one additional acrylic monomer chosen from (meth)acrylic acid and the alkyl (meth)acrylates of formula (IX) defined below, and their salts, in order to form said insoluble backbone; and

of at least one silicone macromonomer comprising an end group which can be polymerized, as defined above.

Examples of suitable main acrylic monomer include, but are not limited to, methyl acrylate, methyl methacrylate, ethyl acrylate, ethyl methacrylate, n-propyl acrylate, n-propyl methacrylate, isopropyl acrylate, isopropyl methacrylate, and mixtures thereof. In at least one embodiment,the at least one main acrylic monomer may be chosen from methyl acrylate, methyl methacrylate, and ethyl methacrylate.

The additional acrylic monomers may be chosen from:

(meth)acrylic acid and its salts,

(meth)acrylates of formula (XI) and their salts:

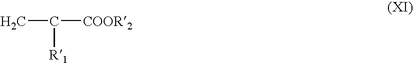

in which:

R′₁ is chosen from hydrogen and methyl;

R′₂ is chosen from:
linear or branched alkyl groups comprising from 1 to 6 carbon atoms, said groups comprising, in their chain, at least one oxygen atom and/or comprising at least one substituent chosen from

—OH, halogen atoms (F, Cl, Br, and I) and —NR′R″, in which R′ and R″, which may be identical or different, are chosen from linear or branched C₁-C₃ alkyls;
cyclic alkyl groups comprising from 3 to 6 carbon atoms, it being possible for said groups to comprise, in their chain, at least one oxygen atom and/or it being possible for said groups to comprise at least one substituent chosen from OH and halogen atoms (F, Cl, Br, and I); and
mixtures-thereof.

Examples of suitable R′₂ groups include, but are not limited to, methoxyethyl, ethoxyethyl, trifluoroethyl, 2-hydroxyethyl, 2-hydroxypropyl, dimethylaminoethyl, diethylaminoethyl, and dimethylaminopropyl groups.

Non-limiting examples of additional acrylic monomers include (meth)acrylic acid, methoxyethyl (meth)acrylate, ethoxyethyl (meth)acrylate, trifluoroethyl methacrylate, dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate, their salts, and mixtures thereof. In at least one embodiment,the at least one additional acrylic monomer may be chosen from acrylic acid and methacrylic acid.

The macromonomers may comprise, at one of the ends of the chain, an end group which can be polymerized and which is capable of reacting during the polymerization with the acrylic monomers and optionally the additional vinyl monomers, in order to form the side chains of the grafted ethylenic polymer. The end group which can be polymerized may be chosen from vinyl groups, (meth)acrylate groups, and (meth)acryloyloxy groups. In at least one embodiment, the end group may be a (meth)acrylate group.

The macromonomers may be chosen from macromonomers, the homopolymer of which has a glass transition temperature (Tg) of less than or equal to 25° C., for example, ranging from −100° C. to 25° C., or from −80° C. to 0° C.

The macromonomers may have a weight-average molecular mass of greater than or equal to 200, for example, greater than or equal to 300, greater than or equal to 500, or greater than 600.

In at least one embodiment of the present disclosure, the macromonomers may have a weight-average molecular mass (Mw) ranging from 200 to 100 000, for instance, from 500 to 50 000, from 800 to 20 000, from 800 to 10 000, or from 800 to 6000.

In the present disclosure, the weight-average (Mw) and number-average (Mn) molar masses are determined by gel permeation liquid chromatography (solvent THF, calibration curve drawn up with linear polystyrene standards, refractometric detector).

Non-limiting examples of carbon macromonomers include:

(i) linear or branched C₈-C₂₂ alkyl (meth)acrylate homopolymers and copolymers comprising an end group which can be polymerized chosen from vinyl groups and (meth)acrylate groups, for example, poly(2-ethylhexyl acrylate) macromonomers comprising a mono(meth)acrylate end; poly(dodecyl acrylate) and poly(dodecyl methacrylate) macromonomers comprising a mono(meth)acrylate end; and poly(stearyl acrylate) and poly(stearyl methacrylate) macromonomers comprising a mono(meth)acrylate end.

Such macromonomers are discussed, for example, in European Patent Nos. 0 895 467 and 0 964 459 and in the article by Gillman K. F., Polymer Letters, 5, 477-481 (1967).

Additional examples of suitable macromonomers include macromonomers based on poly(2-ethylhexyl acrylate) and macromonomers based on poly(dodecyl acrylate) comprising a mono(meth)acrylate end.

(ii) polyolefins comprising an end group comprising ethylenic unsaturation, for example, a (meth)acrylate end group. Examples of such polyolefins include, but are not limited to, polyethylene macromonomers, polypropylene macromonomers, polyethylene/polypropylene copolymer macromonomers, polyethylene/polybutylene copolymer macromonomers, polyisobutylene macromonomers, polybutadiene macromonomers, polyisoprene macromonomers, polybutadiene macromonomers, and poly(ethylene/butylene)-polyisoprene macromonomers, it being understood that these macromonomers comprise a (meth)acrylate end group.

Such macromonomers are disclosed, for example, in U.S. Pat. No. 5,625,005, which mentions ethylene/butylene and ethylene/propylene macromonomers comprising a reactive (meth)acrylate end group.

Mention may be made of poly(ethylene/butylene) methacrylate, such as that sold under the name Kraton Liquid L-1253 by Kraton Polymers.

Mention may be made, as silicone macromonomers, of polydimethylsiloxanes possessing a mono(meth)acrylate end group, for example, those of formula (XII):

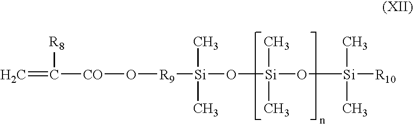

in which:

R₈ is chosen from hydrogen and methyl;

R₉ is chosen from divalent hydrocarbon groups comprising from 1 to 10 carbon atoms and optionally comprising one or two —O— ether bonds;

R₁₀ is chosen from alkyl groups comprising from 1 to 10 carbon atoms, for example, from 2 to 8 carbon atoms; and

n is an integer ranging from 1 to 300, for example, from 3 to 200, or from 5 to 100.

Examples of silicone macromonomers include, but are not limited to, monomethacryloyloxypropylpolydimethylsiloxanes, such as those sold under the name PS560-K6 by United Chemical Technologies Inc. (UCT) or under the name MCR-M17 by Gelest Inc.

The polymerized macromonomer (constituting the side chains of the grafted polymer) may represent from 0.1 to 15% by weight of the total weight of the polymer, for example, from 0.2 to 10% by weight, or from 0.3 to 8% by weight.

Non-limiting examples of grafted ethylenic polymers dispersed in a nonsilicone liquid fatty phase include those obtained by polymerization:

of methyl acrylate and of the polyethylene/polybutylene macromonomer comprising a methacrylate end group (for example, Kraton L-1253), for example, in a solvent chosen from isododecane, isononyl isononanoate, octyldodecanol, diisostearyl malate, and C₁₂-Cl₅ alkylbenzoates (such as Finsolv TN);

of methoxyethyl acrylate and of the polyethylene/polybutylene macromonomer comprising a methacrylate end group (for example, Kraton L-1253), in a solvent chosen, for example, from isododecane;

of methyl acrylate/methyl methacrylate monomer and of the polyethylene/polybutylene macromonomer comprising a methacrylate end group (for example, Kraton L-1253), in a solvent chosen, for example, from isododecane;

of methyl acrylate/acrylic acid monomer and of the polyethylene/polybutylene macromonomer comprising a methacrylate end group (for example, Kraton L-1253), in a solvent chosen, for example, from isododecane;

of methyl acrylate/dimethylaminoethyl methacrylate monomer and of the polyethylene/polybutylene macromonomer comprising a methacrylate end group (for example, Kraton L-1253), in a solvent chosen, for example, from isododecane; or

of methyl acrylate/2-hydroxyethyl methacrylate monomer and of the polyethylene/polybutylene macromonomer possessing a methacrylate end group (for example, Kraton L-1253), in a solvent chosen, for example, from isododecane.

Examples of grafted acrylic polymers dispersed in a silicone liquid fatty phase include, but are not limited to, those obtained by polymerization:

of methyl acrylate and of the monomethacryloyloxypropylpolydimethylsiloxane macromonomer having a weight-average molecular weight ranging from 800 to 6000, for example, decamethylcyclopentasiloxane and phenyl trimethicone; or

of methyl acrylate, of acrylic acid, and of the monomethacryloyloxypropylpolydimethylsiloxane macromonomer having a weight-average molecular weight ranging from 800 to 6000, for instance, decamethylcyclopentasiloxane and phenyl trimethicone.

The grafted polymer may have a weight-average molecular mass (Mw) ranging from 10 000 to 300 000, for example, ranging from 20 000 to 200 000, or from 25 000 to 150 000.

By virtue of the abovementioned characteristics, in a given dispersion organic medium, the polymers may have the ability to withdraw into themselves, thus forming particles of substantially spherical shape, with the side chains opened out on the perimeter of these particles, which side chains may ensure the stability of these particles. Such particles, resulting from the characteristics of the grafted polymer, may have the distinguishing feature of not agglomerating in said medium, and thus, of self-stabilizing and of forming a dispersion of polymer particles exhibiting improved stability.

For example, the grafted ethylenic polymers of the dispersion may form nanometric particles with a mean size ranging from 10 to 400 nm, for example, from 20 to 200 nm.

Due to this very small size, the dispersed particles of grafted polymer may exhibit improved stability, and thus, may be less susceptible to forming agglomerates.

The dispersion of grafted polymer may thus be a stable dispersion and may not form sediments when it is placed for a prolonged period of time (for example, 24 hours) at ambient temperature (25° C.).

In at least one embodiment, the dispersion of grafted polymer particles may have a dry matter content (or solids content) of polymer ranging from 40% to 70% by weight of dry matter, for example, ranging from 45% to 65% by weight.

The dispersion of grafted polymer particles may be prepared by a process comprising a stage of radical copolymerization, in an organic polymerization medium, of at least one acrylic monomer as defined above with at least one macromonomer as defined above.

As indicated above, the dispersing liquid organic medium may be identical to or different from the polymerization medium.

Conventionally, the copolymerization may be carried out in the presence of a polymerization initiator. The polymerization initiators may be radical initiators. Generally, such a polymerization initiator may be chosen from organic peroxide compounds, such as dilauroyl peroxide, dibenzoyl peroxide, and tert-butyl peroxy-2-ethylhexanoate, and diazo compounds, such as azobisisobutyronitrile and azobisdimethylvaleronitrile.

The reaction may also be initiated using photoinitiators, with radiation, such as UV radiation and neutrons, and with plasma.

Generally, in order to carry out this process, at least a portion of the organic polymerization medium, a portion of the acrylic and/or additional vinyl monomers, which will constitute, after polymerization, the insoluble backbone, all the macromonomer (which will constitute the side chains of the polymer), and a portion of the polymerization initiator may be introduced into a reactor with a size appropriate to the amount of polymer which will be produced. At this stage of introduction, the reaction medium forms a relatively homogeneous medium.

The reaction medium is subsequently stirred and heated up to a temperature in order to obtain polymerization of the monomers and macromonomers. After a certain period of time, the initially homogeneous and clear medium results in a dispersion with a milky appearance. A mixture comprising the remaining portion of monomers and of the polymerization initiator is subsequently added. After an appropriate time, during which the mixture is heated with stirring, the medium stabilizes in the form of a milky dispersion, the dispersion comprising particles of polymers stabilized in the medium in which they were created, said stabilization being due to the presence, in the polymer, of side chains which are soluble in said dispersing medium.

The grafted polymer may be present in the composition according to the present disclosure in an amount of dry matter (or active material) ranging from 1 to 70% by weight, with respect to the total weight of the composition, for example, from 5 to 60% by weight, from 6 to 45%, or from 8 to 40% by weight.

Linear Ethylenic Sequential Polymer

The composition according to the present disclosure may comprise, as a film-forming agent, at least one linear ethylenic sequential polymer, referred to herein as a “sequential polymer”, with a structure as described below.

The term “‘sequential’ polymer” is understood to mean a polymer comprising at least 2 separate sequences, for example, at least 3 separate sequences.

The polymer may have a linear structure. In contrast, a polymer with a nonlinear structure is, for example, a polymer with a structure chosen from branched, star, grafted, and other structures.

In at least one embodiment, the sequential polymer may be devoid of styrene. As used herein, the term “polymer devoid of styrene” is understood to mean a polymer comprising less than 10% by weight styrene monomers, with respect to the total weight of the polymer, for instance, less than 5% by weight, less than 2% by weight, or less than 1% by weight, or being devoid of styrene monomers, such as styrene and styrene derivatives, for example, methylstyrene, chlorostyrene, and chloromethylstyrene.

According to one embodiment, the sequential polymer may comprise at least one first sequence and at least one second sequence having different glass transition temperatures (Tg), said first and second sequences being connected to one another via an intermediate sequence comprising at least one constituent monomer of the first sequence and at least one constituent monomer of the second sequence.

As used herein, the term “‘at least’ one sequence” is understood to mean one or more sequences.

The intermediate sequence may be a sequence comprising at least one constituent monomer of the first sequence and at least one constituent monomer of the second sequence of the polymer, which makes it possible to “compatibilize” these sequences.

It is to be understood that the terms “first” and “second” sequences do not in any way condition the order of the said sequences (or blocks) in the structure of the sequential polymer.

The first and second sequences of the sequential polymer may be incompatible with one another.

As used herein, the term “sequences incompatible with one another” is understood to mean that the blend comprising the polymer corresponding to the first sequence and the polymer corresponding to the second sequence is immiscible in the predominant organic liquid by weight of the liquid fatty phase at ambient temperature (25° C.) and atmospheric pressure (10⁵ Pa), and at a concentration of the blend of polymers of greater than or equal to 5% by weight, with respect to the total weight of the mixture (polymers and solvent), it being understood that:

i) said polymers are present in the blend in a content such that the respective ratio by weight ranges from 10/90 to 90/10, and that

ii) each of the polymers corresponding to the first and second sequences has a (weight- or number)-average molecular mass equal to that of the sequential polymer±15%.

In the case where the liquid fatty phase comprises a mixture of organic liquids, and under the assumption of two or more organic liquids present in identical proportions by weight, said blend of polymers is immiscible in at least one of them.

In the case where the liquid fatty phase comprises a single organic liquid, the latter is the predominant organic liquid.

In at least one embodiment, the sequential polymer may not comprise silicon atoms in its backbone. As used herein, the term “backbone” is understood to mean the main chain of the polymer, in contrast to the pendent side chains.

The sequential polymer may be insoluble in water or in a mixture of water and linear or branched lower monoalcohols comprising from 2 to 5 carbon atoms, such as ethanol, isopropanol, and n-propanol, without modification of pH, at a content of active material of at least 1% by weight, and at ambient temperature (25° C.).

In one embodiment of the present disclosure, the sequential polymer may not be an elastomer. As used herein, the term “non-elastomeric polymer” is understood to mean a polymer which, when it is subjected to a stress targeted at drawing it (for example, by 30% relative to its initial length), does not return to a length substantially identical to its initial length when the stress ceases.

More specifically, as used herein, the term “non-elastomeric polymer” denotes a polymer having an instantaneous recovery Rᵢ<50% and a delayed recovery R₂ₕ<70% after having undergone an elongation of 30%. In one embodiment, the sequential polymer may have an Rᵢ<30% and R₂ₕ<50%.

The non-elastomeric nature of the polymer disclosed herein is determined according to the following protocol:

A polymer film is prepared by pouring a solution of the polymer into a Teflon-treated matrix and then drying for 7 days in surroundings controlled at 23±5° C. and 50±10% relative humidity.

A film with a thickness of approximately 100 μm is then obtained, from which rectangular test specimens having a width of 15 mm and a length of 80 mm are cut (for example, with a hollow punch).

A tensile stress is applied to this sample using a device sold under the name Zwick, under the same temperature and humidity conditions as for the drying.

The test specimens are drawn at a rate of 50 mm/min and the distance between the clamping jaws is 50 mm, which corresponds to the initial length (I₀) of the test specimen.

The instantaneous recovery Rᵢ is determined in the following way:

the test specimen is drawn by 30% (εₘₐₓ), that is to say approximately 0.3 times its initial length (I₀),

the stress is released by applying a return rate equal to the tensioning rate, i.e., 50 mm/min, and the residual elongation of the test specimen is measured as a percentage, after returning to zero stress (εᵢ).

The instantaneous recovery in % (Rᵢ) is given by the formula below:
Rᵢ=((εₘₐₓ−εᵢ)/εₘₐₓ)×100.

To determine the delayed recovery, the residual elongation of the test specimen is measured as a percentage (ε₂ₕ), 2 hours after returning to zero stress.

The delayed recovery in % (R₂ₕ) is given by the formula below:
R₂ₕ=((εₘₐₓ−ε₂ₕ)/εₘₐₓ)×100.

Purely by way of indication, a polymer according to one embodiment of the present disclosure may have an instantaneous recovery Rᵢ of 10% and a delayed recovery R₂ₕ of 30%.

In at least one embodiment, the sequential polymer may, for example, have a polydispersity index I of greater than 2 (for example, ranging from 2 to 9), of greater than or equal to 2.5 (for example, ranging from 2.5 to 8), or of greater than or equal to 2.8 (for example, ranging from 2.8 to 6).

The polydispersity index I of the sequential polymer is equal to the ratio of the weight-average mass Mw to the number-average mass Mn.

The weight-average molar masses (Mw) and the number-average molar masses (Mn) may be determined by gel permeation liquid chromatography (solvent THF, calibration curve drawn up with linear polystyrene standards, refractometric detector).

The weight-average mass (Mw) of the sequential polymer may be less than or equal to 300 000; and may range, for example, from 35 000 to 200 000, or from 45 000 to 150 000.

The number-average mass (Mn) of the sequential polymer may be less than or equal to 70 000; and may range, for example, from 10 000 to 60 000, or from 12 000 to 50 000.

Each sequence or block of the sequential polymer may result from one type of monomer or from several different types of monomers. This means that each sequence may comprise a homopolymer or a copolymer; this copolymer being chosen from, for example, random and alternating copolymers.

In one embodiment, the intermediate sequence comprising at least one constituent monomer of the first sequence and at least one constituent monomer of the second sequence of the sequential polymer may be a random polymer.

In another embodiment, the intermediate sequence may essentially comprise constituent monomers of the first sequence and of the second sequence.

As used herein, the term “essentially” is understood to mean at least 85%, for example, at least 90%, at least 95%, or up to 100%.

The intermediate sequence may have a glass transition temperature Tg between the glass transition temperatures of the first and second sequences.

The glass transition temperatures indicated for the first and second sequences can be theoretical Tg values determined from the theoretical Tg values of the constituent monomers of each of the sequences, which can be found in a reference handbook, such as the Polymer Handbook, 3rd ed., 1989, John Wiley, according to the following relationship, referred to as the Fox law:

$$1 / Tg = {\sum }_{i} ( {ϖ}_{l} / {Tg}_{l} ) .$$

wherein \overline{ωᵢ} is the mass fraction of the monomer i in the sequence under consideration, and

Tgᵢ is the glass transition temperature of the homopolymer of the monomer i.

Unless otherwise indicated, the Tg values indicated for the first and second sequences in the present patent application are theoretical Tg values.

The difference between the glass transition temperatures of the first and second sequences generally may be greater than 10° C., for example, greater than 20° C., or greater than 30° C.

For example, the first sequence of the sequential polymer may be chosen from:

a) a sequence having a Tg of greater than or equal to 40° C.,

b) a sequence having a Tg of less than or equal to 20° C., and

c) a sequence having a Tg ranging from 20 to 40° C.,

and the second sequence chosen from a category a), b), or c) different from the first sequence.

As used herein, the expression: “of between . . . and . . . ” is intended to denote a range of values, the limits of which mentioned are excluded, and “from . . . to . . . ” and “ranging from . . . to . . . ” is intended to denote a range of values, the limits of which are included.

a) Sequence having a Tq of Greater than or Equal to 40° C.

The sequence having a Tg of greater than or equal to 40° C. may have, for example, a Tg ranging from 40 to 150° C., of greater than or equal to 50° C., for example, ranging from 50° C. to 120° C., or of greater than or equal to 60° C., for example, ranging from 60° C. to 120° C.

The sequence having a Tg of greater than or equal to 40° C. may be chosen from homopolymers and copolymers.

In the case where this sequence is a homopolymer, it may result from monomers such that the homopolymers prepared from these monomers have glass transition temperatures of greater than or equal to 40° C. This first sequence may be a homopolymer comprising a single type of monomer (the Tg of the corresponding homopolymer of which is greater than or equal to 40° C.).

In the case where the first sequence is a copolymer, it may result, in all or in part, from at least one monomer, the nature and the concentration of which are chosen so that the Tg of the resulting copolymer is greater than or equal to 40° C. The copolymer may, for example, comprise:

monomers, the corresponding homopolymer of which has a Tg value of greater than or equal to 40° C. (for example, a Tg ranging from 40 to 150° C.), of greater than or equal to 50° C. (for example, a Tg ranging from 50° C. to 120° C.), or of greater than or equal to 60° C. (for example, a Tg ranging from 60° C. to 120° C.), and

monomers, the corresponding homopolymer of which has a Tg value of less than 40° C., chosen from monomers having a Tg ranging from 20 to 40° C. and/or monomers having a Tg of less than or equal to 20° C. (for example, a Tg ranging from −100 to 20° C.), of less than 15° C. (for example, ranging from −80° C. to 15° C.), or of less than 10° C. (for example, ranging from −50° C. to 0° C.), as described below.

The monomers, the homopolymers of which have a glass transition temperature of greater than or equal to 40° C., may be chosen from main monomers including, but not limited to:

methacrylates of formula CH₂═C(CH₃)—COOR₁,

in which R₁ is chosen from unsubstituted, linear or branched alkyl groups comprising from 1 to 4 carbon atoms, such as a methyl, ethyl, propyl,and isobutyl groups, and C₄ to C₁₂ cycloalkyl groups,

acrylates of formula CH₂═CH—COOR₂,

in which R₂ is chosen from C₄ to C₁₂ cycloalkyl groups, such as isobornyl acrylate, and tert-butyl groups,

(meth)acrylamides of formula (XIII):

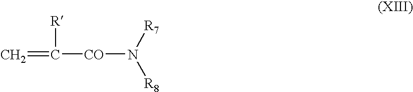

wherein:

R₇ and R₈, which may be identical or different, are chosen from hydrogen and linear or branched C₁ to C₁₂ alkyl groups, such as n-butyl, t-butyl, isopropyl, isohexyl, isooctyl, and isononyl groups, or alternatively, R₇ is hydrogen and R₆ is a 1,1-dimethyl-3-oxobutyl group, and

R′ is chosen from hydrogen and methyl. Examples of such (meth)acrylamide monomers include, but are not limited to, N-butylacrylamide, N-(t-butyl)acrylamide, N-isopropylacrylamide, N,N-dimethylacrylamide, and N,N-dibutylacrylamide,

and mixtures thereof.

In at least one embodiment, the main monomers may be chosen from methyl methacrylate, isobutyl (meth)acrylate, isobornyl (meth)acrylate, and mixtures thereof.

b) Sequence having a Tq of Less than or Equal to 20° C.

The sequence having a Tg of less than or equal to 20° C. may have, for instance, a Tg ranging from −100 to 20° C., of less than or equal to 15° C., for example, ranging from −80° C. to 15° C., or of less than or equal to 10° C., for example, ranging from −50° C. to 0° C.

The sequence having a Tg of less than or equal to 20° C. may be chosen from homopolymers and copolymers.

In the case where this sequence is a homopolymer, it may result from monomers such that the homopolymers prepared from these monomers have glass transition temperatures of less than or equal to 20° C. This second sequence may be a homopolymer comprising a single type of monomer (the Tg of the corresponding homopolymer of which is less than or equal to 20° C.).

In the case where the sequence having a Tg of less than or equal to 20° C. is a copolymer, it may result, in all or in part, from at least one monomer, the nature and the concentration of which are chosen so that the Tg of the resulting copolymer is less than or equal to 20° C.

The copolymer may, for example, comprise

at least one monomer, the corresponding homopolymer of which has a Tg of less than or equal to 20° C. (for example, a Tg ranging from −100° C. to 20° C.), of less than 15° C. (for example, a Tg ranging from −80° C. to 15° C.), or of less than 10° C. (for example, a Tg ranging from −50° C. to 0° C.), and

at least one monomer, the corresponding homopolymer of which has a Tg of greater than 20° C., such as monomers having a Tg of greater than or equal to 40° C. (for example, a Tg ranging from 40 to 150° C.), of greater than or equal to 50° C. (for example, a Tg ranging from 50° C. to 120° C.), or of greater than or equal to 60° C. (for example, ranging from 60° C. to 120° C.), and/or the monomers having a Tg ranging from 20 to 40° C., as described above.

In at least one embodiment, the sequence having a Tg of less than or equal to 20° C. may be chosen from homopolymers.

The monomers, the homopolymer of which has a Tg of less than or equal to 20° C. may be chosen from main monomers including, but not limited to:

acrylates of formula CH₂═CHCOOR₃,

wherein R₃ is chosen from unsubstituted, linear or branched C₁ to C₁₂ alkyl groups, with the exception of the tert-butyl group, in which at least one heteroatom chosen from O, N, and S is optionally inserted,

methacrylates of formula CH₂═C(CH₃)—COOR₄,

wherein R₄ is chosen from unsubstituted, linear or branched C₆ to C₁₂ alkyl groups in which at least one heteroatom chosen from O, N, and S is optionally inserted,

vinyl esters of formula R₅—CO—O—CH═CH₂,

wherein R₅ is chosen from linear or branched C₄ to C₁₂ alkyl groups,

C₄ to C₁₂ alkyl vinyl ethers,

N—(C₄ to C₁₂ alkyl)acrylamides, such as N-octylacrylamide, and

mixtures thereof.

In at least one embodiment, the main monomers for the sequence having a Tg of less than or equal to 20° C. may be chosen from alkyl acrylates, the alkyl chain of which comprises from 1 to 10 carbon atoms, with the exception of the tert-butyl group, such as methyl acrylate, isobutyl acrylate, 2-ethylhexyl acrylate, and mixtures thereof.

c) Sequence having a Tq Ranging from 20 to 40° C.

The sequence which has a Tg ranging from 20 to 40° C. may be chosen from homopolymers and copolymers.

In the case where this sequence is a homopolymer, it may result from monomers (or main monomers), the corresponding homopolymer of which has a glass transition temperature ranging 20 and 40° C. This first sequence may be a homopolymer comprising a single type of monomer (the Tg of the corresponding homopolymer of which ranges from 20° C to 40° C.).

Monomers, the corresponding homopolymer of which has a glass transition temperature ranging from 20 to 40° C., may be chosen from n-butyl methacrylate, cyclodecyl acrylate, neopentyl acrylate, isodecylacrylamide, and mixtures thereof.

In the case where the sequence having a Tg ranging from 20 to 40° C. is a copolymer, it may results, all or in part, from at least one monomer (or main monomer), the nature and the concentration of which are chosen so that the Tg of the resulting copolymer ranges from 20 to 40° C.

In at least one embodiment, the sequence having a Tg ranging from 20 to 40° C. may be chosen from copolymers resulting, all or in part, from:

main monomers, the corresponding homopolymer of which has a Tg of greater than or equal to 40° C. (for example, a Tg ranging from 40° C. to 150° C.), of greater than or equal to 50° C. (for example, a Tg ranging from 50 to 120° C.), or of greater than or equal to 60° C. (for example, a Tg ranging from 60° C. to 120° C.), as described above, and/or

main monomers, the corresponding homopolymer of which has a Tg of less than or equal to 20° C. (for example, a Tg ranging from −100 to 20° C.), of less than or equal to 15° C. (for example, a Tg ranging from −80° C. to 15° C.) or of less than or equal to 10° C. (for example, ranging from −50° C. to 0° C.), as described above,

said monomers being chosen so that the Tg of the copolymer forming the first sequence ranges from 20 to 40° C.

Such main monomers may be chosen, for example, from methyl methacrylate, isobornyl acrylate, isobornyl methacrylate, butyl acrylate, 2-ethylhexyl acrylate, and mixtures thereof.

In one embodiment, the second sequence having a Tg of less than or equal to 20° C. is present in the polymer in an amount ranging from 10 to 85% by weight of the polymer, for instance, from 20 to 70%, or from 20 to 50%.

Each of the sequences may nevertheless comprise a minor proportion of at least one constituent monomer of the other sequence. Thus, the first sequence may comprise at least one constituent monomer of the second sequence, and vice versa.

Each of the first and/or second sequences of the sequential polymer may comprise, in addition to the monomers described above, at least one other monomer, known as an additional monomer, different from the main monomers mentioned above.

The nature and amount of the at least one additional monomer are chosen so that the sequence in which they occur has the desired glass transition temperature.

This additional monomer may be chosen, for example, from:

a) hydrophilic monomers, such as:
monomers comprising at least one ethylenic unsaturation comprising at least one functional group chosen from carboxylic and sulphonic acid functional groups, for example acrylic acid, methacrylic acid, crotonic acid, maleic anhydride, itaconic acid, fumaric acid, maleic acid, acrylamidopropanesulphonic acid, vinylbenzoic acid, vinylphosphonic acid, and the salts thereof,
monomers comprising at least one ethylenic unsaturation comprising at least one tertiary amine functional group, such as 2-vinylpyridine, 4-vinylpyridine, dimethylaminoethyl methacrylate, diethylaminoethyl methacrylate, dimethylaminopropylmethacrylamide, and the salts thereof,
methacrylates of formula CH₂═C(CH₃)—COOR₆,

in which:

R₆ is chosen from linear or branched alkyl groups comprising from 1 to 4 carbon atoms, such as a methyl, ethyl, propyl, and isobutyl groups, said alkyl groups being substituted by at least one substituent chosen from hydroxyl groups (such as 2-hydroxypropyl methacrylate and 2-hydroxyethyl methacrylate) and halogen atoms (such as Cl, Br, I, and F), such as trifluoroethyl methacrylate,

methacrylates of formula CH₂═C(CH₃)—COOR₉,

in which:

R₉ is chosen from linear or branched C₆ to C₁₂ alkyl groups in which at least one heteroatom chosen from O, N, and S is optionally inserted, said alkyls group being substituted by at least one substituent chosen from hydroxyl groups and halogen atoms (Cl, Br, I, and F),

acrylates of formula CH₂═CHCOOR₁₀,

in which

R₁₀ is chosen from linear or branched C₁ to C₁₂ alkyl groups substituted by at least one substituent chosen from hydroxyl groups and halogen atoms (Cl, Br, I, and F), such as 2-hydroxypropyl acrylate and 2-hydroxyethyl acrylate; (C₁-C₁₂)alkyl-O-POEs (polyoxyethylenes) comprising from 5 to 30 repetitions of the oxyethylene unit, for example methoxy-POE; and polyoxyethylene groups comprising from 5 to 30 ethylene oxide units,

b) monomers comprising ethylenic unsaturation comprising at least one silicon atom, such as methacryloyloxypropyltrimethoxysilane or methacryloyloxypropyltris(trimethylsiloxy)silane, and

mixtures thereof.

In one embodiment, the at least one additional monomer may be chosen from acrylic acid, methacrylic acid, trifluoroethyl methacrylate, and mixtures thereof.

According to at least one embodiment, the sequential polymer may be chosen from nonsilicone polymers, i.e., polymers devoid of silicon atoms.

The at least one additional monomer may be present in the first and/or second sequences an amount of less than or equal to 30% by weight, for example, from 1 to 30% by weight, from 5 to 20% by weight, or from 7 to 15% by weight, of the total weight of the first and/or second sequences.

In one embodiment, each of the first and second sequences may comprises at least one monomer chosen from (meth)acrylic acid esters, and optionally, at least one monomer chosen from (meth)acrylic acid, and mixtures thereof.

In another embodiment, each of the first and second sequences of the sequential polymer may result from at least one monomer chosen from acrylic acid and (meth)acrylic acid esters, and optionally, from at least one monomer chosen from (meth)acrylic acid, and mixtures thereof.

The sequential polymer may be obtained by radical solution polymerization according to the following preparation process:

a portion of the polymerization solvent is introduced into a suitable reactor and is heated until the temperature appropriate for the polymerization is reached (typically ranging from 60 to 120° C.),

once this temperature has been reached, the constituent monomers of the first sequence are introduced in the presence of a portion of the polymerization initiator,

after a time T corresponding to a maximum degree of conversion of 90%, the constituent monomers of the second sequence and the other portion of the initiator are introduced,

the mixture is allowed to react for a time T′ (ranging from 3 to 6 h), at the end of which the mixture is brought back to ambient temperature, and

the polymer is obtained in solution in the polymerization solvent.

As used herein, the term “polymerization solvent” is understood to mean a solvent or a mixture of solvents. The polymerization solvent may be chosen, for example, from ethyl acetate; butyl acetate; alcohols, such as isopropanol and ethanol; aliphatic alkanes, such as isododecane; and mixtures thereof. In at least one embodiment, the polymerization solvent may be chosen from mixtures of butyl acetate and isopropanol and mixtures of butylacetate and isododecane.

According to one embodiment, the sequential polymer may comprise a first sequence having a Tg of greater than or equal to 40° C., as described above in a), and a second sequence having a Tg of less than or equal to 20° C., as described above in b).

In another embodiment, the first sequence having a Tg of greater than or equal to 40° C. may be chosen from copolymers resulting from monomers, the corresponding homopolymer of which has a glass transition temperature of greater than or equal to 40° C., such as the monomers described above.

In a further embodiment, the second sequence having a Tg of less than or equal to 20° C. may be chosen from homopolymers resulting from monomers, the corresponding homopolymer of which has a glass transition temperature of less than or equal to 20° C., such as the monomers described above.

The sequence having a Tg of greater than or equal to 40° C. may be present in the sequential polymer in an amount ranging from 20 to 90% by weight of the polymer, for example, from 30 to 80%, or from 50 to 70%.

The sequence having a Tg of less than or equal to 20° C. may be present in the sequential polymer in an amount ranging from 5 to 75% by weight of the polymer, for example, from 15 to 50%, or from 25 to 45%.

In at least one embodiment of the present disclosure, the sequential polymer may comprise:

a first sequence with a Tg of greater than or equal to 40° C., for example, ranging from 85 to 115° C., which is an isobornyl acrylate/isobutyl methacrylate copolymer,

a second sequence with a Tg of less than or equal to 20° C., for example ranging from −85 to −55° C., which is a 2-ethylhexyl acrylate homopolymer, and

an intermediate sequence which is an isobornyl acrylate/isobutyl methacrylate/2-ethylhexyl acrylate random copolymer.

According to another embodiment, the sequential polymer may comprise a first sequence having a glass transition temperature (Tg) ranging from 20 to 40° C., as described above in c), and a second sequence having a glass transition temperature of less than or equal to 20° C., as described above in b), or a glass transition temperature of greater than or equal to 40° C., as described in a) above.

The first sequence having a Tg ranging from 20 to 40° C. may be present in the sequential polymer in an amount ranging from 10 to 85% by weight of the polymer, for example, from 30 to 80%, or from 50 to 70%.

When the second sequence is a sequence having a Tg of greater than or equal to 40° C., it may be present in the sequential polymer in an amount ranging from 10 to 85% by weight of the polymer, for instance, from 20 to 70%, or from 30 to 70%.

When the second sequence is a sequence having a Tg of less than or equal to 20° C., it may be present in the sequential polymer in an amount ranging from 10 to 85% by weight of the polymer, for example, from 20 to 70%, or from 20 to 50%.

In one embodiment, the first sequence having a Tg ranging from 20 to 40° C. may be chosen from copolymers resulting from monomers, the corresponding homopolymer of which has a Tg of greater than or equal to 40° C. and from monomers, the corresponding homopolymer of which has a Tg of less than or equal to 20° C.

According to another embodiment, the second sequence having a Tg of less than or equal to 20° C. or having a Tg of greater than or equal to 40° C. may be chosen from homopolymers.

According to a further embodiment of the present disclosure, the sequential polymer may comprise:

a first sequence with a Tg ranging from 20 to 40° C., for example, having a Tg from 21 to 39° C., which is an isobornyl acrylate/isobutyl methacrylate/2-ethylhexyl acrylate copolymer,

a second sequence with a Tg of less than or equal to 20° C., for example ranging from −65 to −35° C., which is a methyl methacrylate homopolymer, and

an intermediate sequence which is an isobornyl acrylate/isobutyl methacrylate/2-ethylhexyl acrylate random copolymer.

According to this embodiment, the sequential polymer may comprise:

a first sequence with a Tg of greater than or equal to 40° C., for example, ranging from 85 to 115° C., which is an isobornyl methacrylate/isobutyl methacrylate copolymer,

a second sequence with a Tg of less than or equal to 20° C., for example, ranging from −35 to −5° C., which is an isobutyl acrylate homopolymer, and

an intermediate sequence which is an isobornyl methacrylate/isobutyl methacrylate/isobutyl acrylate random copolymer.

According to yet another embodiment, the sequential polymer may comprise:

a first sequence with a Tg of greater than or equal to 40° C., for example, ranging from 60 to 90° C., which is an isobornyl acrylate/isobutyl methacrylate copolymer,

a second sequence with a Tg of less than or equal to 20° C., for example, ranging from −35 to −5° C., which is an isobutyl acrylate homopolymer, and

an intermediate sequence which is an isobornyl acrylate/isobutyl methacrylate/isobutyl acrylate random copolymer.

The at least one film-forming polymer may be of any chemical type and may be chosen from:

a) fat-soluble and amorphous homopolymers and copolymers of olefins, cycloolefins, butadiene, isoprene, styrene, vinyl ethers, esters, and amides; esters and amides of (meth)acrylic acid comprising at least one linear, branched, or cyclic C₄₋₅₀ alkyl group. In at least one embodiment, the homopolymers and copolymers are amorphous. The fat-soluble homopolymers and copolymers may be obtained from monomers chosen from isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate, and mixtures thereof. Suitable examples of copolymers include, but are not limited to, the alkyl acrylate/cycloalkyl acrylate copolymer sold by Phoenix Chem under the name Giovarez AC-5099 ML, and vinylpyrrolidone copolymers, such as copolymers of a C₂ to C₃₀ alkene, for example, a C₃ to C₂₂ alkene, and combinations thereof. Non-limiting examples of VP (vinylpyrrolidone) copolymers which may be used in the present disclosure include VP/vinyl laurate, VP/vinyl stearate, VP/hexadecene, VP/triacontene, VP/acrylic acid/lauryl methacrylate copolymer, and butylated polyvinylpyrrolidone (PVP).

Examples of fat-soluble copolymers include, but are not limited to:

i) grafted silicone-acrylic polymers comprising a silicone backbone and acrylic grafts or comprising an acrylic backbone and silicone grafts, such as the product sold under the name SA 70.5 by 3M and disclosed in U.S. Pat. Nos. 5,725,882, 5,209,924, 4,972,037, 4,981,903, 4,981,902, 5,468,477, and 5,219,560 and European Patent No. 0 388 582;

ii) fat-soluble polymers comprising fluorinated groups belonging to one of the categories described in the above text, for example, Fomblin, those disclosed in U.S. Pat. No. 5,948,393, and the alkyl (meth)acrylate/perfluoroalkyl (meth)acrylate copolymers disclosed in European Patent Nos. 0 815 836 and 5 849 318;

iii) polymers or copolymers resulting from the polymerization or the copolymerization of an ethylenic monomer comprising at least one ethylenic bond, for example, conjugated bonds (or dienes). Examples of polymers or copolymers resulting from the polymerization or the copolymerization of an ethylenic monomer, include, but are not limited to, vinyl, acrylic, and methacrylic copolymers.

In one embodiment, the at least one film-forming agent may be a block copolymer comprising at least one block comprising styrene units and/or units derived from styrene (for example, methylstyrene, chlorostyrene, and chloromethylstyrene). The copolymer comprising at least one styrene block may be chosen from star diblock, star triblock, radial diblock, radial triblock copolymers, star multiblock copolymers, and radial multiblock copolymers. The copolymer comprising at least one styrene block may additionally comprise, for example, at least one block chosen from alkylstyrene (AS) blocks, ethylene/butylene (EB) blocks, ethylene/propylene (EP) blocks, butadiene (B) blocks, isoprene (I) blocks, acrylate (A) blocks, methacrylate (MA) blocks, and combinations thereof. The copolymer comprising at least one block comprising styrene units and/or units derived from styrene may be chosen from diblock and triblock copolymers, for example, those of the polystyrene/polyisoprene and polystyrene/polybutadiene types, such as those sold or manufactured under the name “Luvitol HSB” by BASF, and those of the polystyrene/copoly(ethylene-propylene) type and of the polystyrene/copoly(ethylene/butylene) type, such as those sold or manufactured under the “Kraton” trade mark by Shell Chemical Co. or Gelled Permethyl 99A by Penreco.

Suitable copolymers may include, for example, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS), Kraton D-1107 (SIS), Gelled Permethyl 99A-750, Gelled Permethyl 99A-753-58 (blend of star block polymer and of triblock polymer), Gelled Permethyl 99A-753-59 (blend of star block polymer and of triblock polymer), Versagel 5970 and Versagel 5960 from Penreco (blend of star polymer and of triblock polymer in isododecane).

Styrene-methacrylate copolymers may also be used, such as the polymers sold under the references OS 129880, OS 129881, and OS 84383 from Lubrizol (styrenemethacrylate copolymer).

In one embodiment, the at least one film-forming agent may be chosen from copolymers of vinyl ester (the vinyl group being directly connected to the oxygen atom of the ester group and the vinyl ester having a saturated, linear or branched, hydrocarbon radical of 1 to 19 carbon atoms bonded to the carbonyl of the ester group) and of at least one other monomer which may be a vinyl ester (different from the vinyl ester already present), α-olefins (comprising from 8 to 28 carbon atoms), alkyl vinyl ethers (the alkyl groups of which comprise from 2 to 18 carbon atoms), allyl and methallyl esters (having a saturated, linear or branched, hydrocarbon radical comprising from 1 to 19 carbon atoms bonded to the carbonyl of the ester group).

These copolymers may be partially crosslinked using crosslinking agents which may be chosen from vinyl allyl, and methallyl copolymers, such as tetraallyloxyethane, divinylbenzene, divinyl octanedioate, divinyl dodecanedioate, and divinyl octadecanedioate.

Examples of these copolymers may include, for example, vinyl acetate/allyl stearate copolymer, vinyl acetate/vinyl laurate copolymer, vinyl acetate/vinyl stearate copolymer, vinyl acetate/octadecene copolymer, vinyl acetate/octadecyl vinyl ether copolymer, vinyl propionate/allyl laurate copolymer, vinyl propionate/vinyl laurate copolymer, vinyl stearate/1-octadecene copolymer, vinyl acetate/1-dodecene copolymer, vinyl stearate/ethyl vinyl ether copolymer, vinyl propionate/cetyl vinyl ether copolymer, vinyl stearate/allyl acetate copolymer, vinyl 2,2-dimethyloctanoate/vinyl laurate copolymer, allyl 2,2-dimethylpentanoate/vinyl laurate copolymer, vinyl dimethylpropionate/vinyl stearate copolymer, allyl dimethylpropionate/vinyl stearate copolymer, vinyl propionate/vinyl stearate crosslinked with 0.2% of divinylbenzene, vinyl dimethylpropionate/vinyl laurate crosslinked with 0.2% of divinylbenzene, vinyl acetate/octadecyl vinyl ether crosslinked with 0.2% of tetraallyloxyethane, vinyl acetate/allyl stearate crosslinked with 0.2% of divinylbenzene, vinyl acetate/1-octadecene crosslinked with 0.2% of divinylbenzene, and allyl propionate/allyl stearate crosslinked with 0.2% of divinylbenzene.

Non-limiting examples of fat-soluble film-forming polymers include fat-soluble copolymers such as those resulting from copolymerization of vinyl esters having from 9 to 22 carbon atoms, copolymerization of alkyl acrylates, or copolymerization of methacrylates, said alkyl radicals comprising from 10 to 20 carbon atoms.

Such fat-soluble copolymers may be chosen from copolymers of poly(vinyl stearate), copolymers of poly(vinyl stearate) crosslinked using divinylbenzene, copolymers of diallyl ether, copolymers of diallyl phthalate, copolymers of poly(stearyl (meth)acrylate), of poly(vinyl laurate), copolymers of poly(lauryl (meth)acrylate), it being possible for these poly(meth)acrylates to be crosslinked using ethylene glycol or tetraethylene glycol dimethacrylate.

The fat-soluble copolymers defined above are disclosed, for example, in French Patent Application No. 2 232 303; they may have a weight-average molecular weight ranging from 2000 to 500 000, for example, from 4000 to 200 000.

Further examples of fat-soluble polymers which may be used in the present disclosure, include, but are not limited to, polyalkylenes and copolymers of C₂-C₂₀ alkenes, for example, polybutene.

b) amorphous and fat-soluble polycondensates, and, in at least one embodiment, not comprising donor groups for hydrogen interactions, for example aliphatic polyesters comprising C₄-₅₀ alkyl side chains and polyesters resulting from the condensation of fatty acid dimers, such as polyesters comprising a silicone segment in the form of a sequence, graft, or end group, as defined, for example, in French Patent Application No. 0 113 920.

c) amorphous and fat-soluble polysaccharides comprising alkyl (ether or ester) side chains, for example, alkylcelluloses comprising a saturated or unsaturated and linear or branched C₁ to C₆ alkyl radicals, such as ethylcellulose and propylcellulose.

The at least one film-forming polymer may be chosen from cellulose polymers, such as nitrocellulose, cellulose acetate, cellulose acetate/butyrate, cellulose acetate/propionate, and ethyl cellulose; polyurethanes; acrylic polymers; vinyl polymers; polyvinylbutyrals; alkyd resins; resins resulting from aldehyde condensation products, such as arylsulphonamide-formaldehyde resins, for example, toluenesulphonamide-formaldehyde resin, and arylsulphonamide-epoxy resins.

Further non-limiting examples of suitable film-forming polymers include nitrocellulose RS ⅛ sec.; RS ¼ sec.; ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; and SS 5 sec., sold, for example, by Hercules; the toluenesulphonamide-formaldehyde resins “Ketjentflex MS80” from Akzo, “Santolite MHP”, “Santolite MS 80”, and “Resimpol 80” from Pan Americana, the alkyd resin “Beckosol ODE 230-70-E” from Dainippon, the acrylic resin “Acryloid B66” from Rohm & Haas, and the polyurethane resin “Trixene PR 4127” from Baxenden.

d) silicone polymers, such as silicone gums and silicone resins.

The viscosity of the silicone gums may range from 1000 to 10 000 000 cSt, for example, from 100 000 to 1 000 000 cSt, or from 300 000 to 700 000 cSt, measured according to Standard ASTM D-445. The silicone gum may be chosen from dimethiconols, fluorosilicones, dimethicones, and mixtures thereof. The dimethicones may include, for example, the dimethicones disclosed in U.S. Pat. No. 4,152,416, for instance, those sold under the references SE30, SE33, SE 54, and SE 76.

Examples of silicone gums which may be used according to the present disclosure,include the product sold under the name SE30 by General Electric, the product sold under the name AK 500000 by Wacker, the product sold under the name Silbione 70047 V by Rhodia, the product sold under the names Q2-1401 and Q2-1403 by Dow Corning, and the product sold under the name 761 by Rhône-Poulenc.

The silicone resins may be soluble or swellable in silicone oils. These resins may be crosslinked polyorganosiloxane polymers.

The nomenclature of silicone resins is known under the name of “MDTQ”, the resin being described as a function of the various siloxane monomer units which it comprises, each of the letters “MDTQ” characterizing one type of unit.

The letter M represents the monofunctional unit of formula (CH₃)₃SiO_{1/2}, the silicon atom being connected to just one oxygen atom in the polymer comprising this unit.

The letter D means a difunctional unit (CH₃)₂SiO_{2/2} in which the silicon atom is connected to two oxygen atoms.

The letter T represents a trifunctional unit of formula (CH₃)SiO_{3/2}.

In the M, D, and T units defined above, at least one of the methyl groups may be replaced by an R group which is different from the methyl group, chosen, for example, from hydrocarbon (for example, alkyl) radicals comprising from 2 to 10 carbon atoms, phenyl groups, and hydroxyl groups.

Finally, the letter Q means a tetrafunctional unit SiO_{4/2} in which the silicon atom is bonded to four oxygen atoms, themselves bonded to the remainder of the polymer.

Various resins possessing different properties may be obtained from these various units, the properties of these polymers varying according to the type of monomers (or units), the type and the number of substituted radicals, the length of the polymer chain, the degree of branching, and the size of the pendent chains.

Examples of these silicone resins include, but are not limited to:

siloxysilicates, which may be trimethylsiloxysilicates of formula [(CH₃)₃XSiXO]ₓX(SiO_{4/2})_{y} (MQ units), in which x and y are integers ranging from 50 to 80,

polysilsesquioxanes of formula (CH₃SiO_{3/2})ₓ (T units) in which x is greater than 100 and at least one of the methyl radicals of which may be replaced by an R group as defined above, and

polymethylsilsesquioxanes, which are polysilsesquioxanes in which none of the methyl radicals has been replaced by another group. Such polymethylsilsesquioxanes are disclosed, for example, in U.S. Pat. No. 5,246,694, which is incorporated herein by reference.

Non-limiting examples of commercially available polymethylsilsesquioxane resins include those sold:

by Wacker under the reference Resin MK, such as Belsil PMS MK, a polymer comprising CH₃SiO_{3/2} repeat units (T units) which may also comprise up to 1% by weight of (CH₃)₂SiO_{2/2} units (D units), and which exhibits an average molecular weight of approximately 10 000,

by Shin-Etsu under the references KR-220L, which comprises T units of formula CH₃SiO_{3/2} and have Si—OH (silanol) end groups, under the reference KR-242A, which comprise 98% of T units and 2% of D dimethyl units and have Si—OH end groups, and under the reference KR-251, which comprise 88% of T units and 12% of D dimethyl units and have Si—OH end groups.

Suitable siloxysilicate resins may be chosen from trimethylsiloxysilicate (TMS) resins, optionally in the form of powders. Such resins are sold, for example, under the reference SR1000 by General Electric and under the reference TMS 803 by Wacker. Further examples include trimethylsiloxysilicate resins sold in a solvent, such as cyclomethicone, for sale under the name “KF-7312J” by Shin-Etsu or “DC 749” and “DC 593” by Dow Corning.

e) Silicone polyamides of the polyorganosiloxane type, such as those disclosed in U.S. Pat. Nos. 5,874,069, 5,919,441, 6,051,216, and 5,981,680.

According to the present disclosure, these silicone polymers may belong to the following two families:

1) polyorganosiloxanes comprising at least two groups capable of establishing hydrogen interactions, these two groups being situated in the chain of the polymer, and/or

2) polyorganosiloxanes comprising at least two groups capable of establishing hydrogen interactions, these two groups being situated on grafts or branchings.

The polymers comprising two groups capable of establishing hydrogen interactions in the chain of the polymer may be chosen from polymers comprising at least one unit corresponding to formula (II):

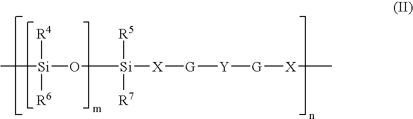

in which:

1) R⁴, R⁵, R⁶, and R⁷, which may be identical or different, are chosen from:

saturated or unsaturated, linear, branched or cyclic, C₁ to C₄₀ hydrocarbon groups which may comprise, in their chain, at least one atom chosen from oxygen, sulphur, and/or nitrogen atoms and which may be substituted, in part or completely, by fluorine atoms,

C₆ to C₁₀ aryl groups, optionally substituted by at least one C₁ to C₄ alkyl group, and

polyorganosiloxane chains which may or may not comprise at least one atom chosen from oxygen, sulphur, and/or nitrogen atoms,

2) the X groups, which may be identical or different, are chosen from linear or branched C₁ to C₃₀ alkylenediyl groups which may comprise, in their chains, at least one atom chosen from oxygen and/or nitrogen atoms,

3) Y is chosen from saturated or unsaturated, C₁ to C₅₀ alkylenes, which may be linear or branched, C₁ to C₅₀ arylenes, C₁ to C₅₀ cycloalkylenes, C₁ to C₅₀ alkylarylenes, and C₁ to C₅₀ arylalkylene divalent groups which may comprise at least one atom chosen from oxygen, sulphur, and/or nitrogen atoms and/or carry, as substituent, at least one entity chosen from fluorine, hydroxyl, C₃ to C₈ cycloalkyls, C₁ to C₄₀ alkyls, C₅ to C₁₀ aryls, and phenyl optionally substituted by 1 to 3 groups chosen from C₁ to C₃ alkyl, C₁ to C₃ hydroxyalkyl, and C₁ to C₆ amino alkyl groups, or alternatively,

4) Y is chosen from groups corresponding to the formula:

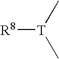

in which:

T is chosen from saturated or unsaturated, linear or branched, trivalent or tetravalent C₃ to C₂₄ hydrocarbon groups which may be optionally substituted by a polyorganosiloxane chain and which may comprise at least one atom chosen from O, N, and S, or alternatively, T is a trivalent atom chosen from N, P, and Al, and

R⁸ is chosen from linear or branched C₁ to C₅₀ alkyl groups and polyorganosiloxane chains, which may comprise at least one group chosen from ester, amide, urethane, thiocarbamate, urea, thiourea, and/or sulphonamide groups, which may or may not be bonded to another chain of the polymer,

5) the G groups, which may be identical or different, are divalent groups chosen from:

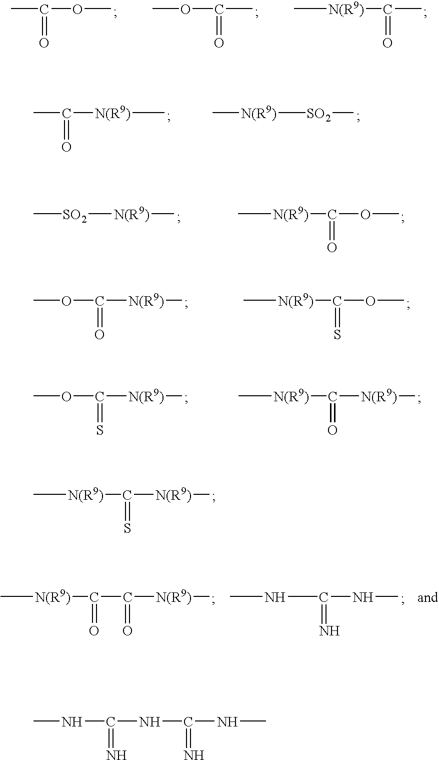

wherein R⁹ is chosen from hydrogen and linear or branched C₁ to C₂₀ alkyl groups, with the proviso that at least 50% of the R⁹ groups of the polymer are hydrogen and that at least two of the G groups of the polymer are a group other than:

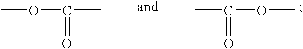

6) n is an integer ranging from 2 to 500, for example, from 2 to 200, and

7) m is an integer ranging from 1 to 1000, for example, from 1 to 700, or from 6 to 200.

According to the present disclosure, 80% of the R⁴, R⁵, R⁶ and R⁶ groups of the polymer are preferably chosen from the methyl, ethyl, phenyl and 3,3,3-trifluoropropyl groups.

According to the present disclosure, Y may be chosen from various divalent groups optionally comprising, in addition, one or two free valences in order to establish bonds with other units of the polymer or copolymer. In at least one embodiment, Y may be chosen from:

a) linear C₁ to C₂₀, for example, C₁ to C₁₀, alkylene groups,

b) branched C₃₀ to C₅₆ alkylene groups which may comprise rings and unconjugated unsaturations,

c) C₅-C₆ cycloalkylene groups,

d) phenylene groups optionally substituted by at least one C₁ to C₄₀ alkyl group,

e) C₁ to C₂₀ alkylene groups comprising from 1 to 5 amide groups,

f) C₁ to C₂₀ alkylene groups comprising at least one substituent chosen from hydroxyl, C₃ to C₈ cycloalkyl, C₁ to C₃ hydroxyalkyl, and C₁ to C₆ alkylamine groups,

g) polyorganosiloxane chains of formula:

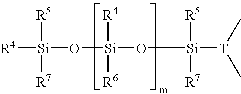

in which R⁴, R⁵, R⁶, and R⁷, T, and m are as defined above, and

h) polyorganosiloxane chains of formula:

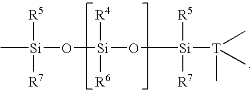

The polyorganosiloxanes of the second family may be polymers comprising at least one unit corresponding to formula (III):

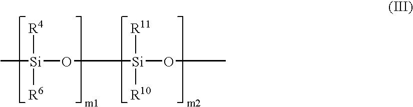

in which:

R⁴ and R⁶, which may be identical or different, are as defined above for formula (II),

R₁₀ is a group as defined above for R⁴ and R⁶, or alternatively, may be chosen from groups of formula —X-G-R¹² in which X and G are as defined above for formula (II) and R¹² is chosen from hydrogen and saturated or unsaturated, linear, branched or cyclic, C₁ to C₅₀ hydrocarbon groups which optionally comprise, in their chain, at least one atom chosen from O, S, and N and which are optionally substituted by at least one entity chosen from fluorine, and/or hydroxyl and phenyl groups optionally substituted by at least one C₁ to C₄ alkyl group,

R¹¹ is chosen from groups of formula —X-G-R¹² in which X, G and R¹² are as defined above,

m₁ is an integer ranging from 1 to 998, and

m₂ is an integer ranging from 2 to 500.

According to the present disclosure, the polymer may be a homopolymer, i.e., a polymer comprising several identical units, for example, units of formula (II) and of formula (III).

According to the present disclosure, the polymer may also be a copolymer comprising several different units of formula (II), i.e., a polymer in which at least one of the R⁴, R⁵, R⁶, R⁷, X, G, Y, m, and n values is different in at least one of the units. The copolymer may also comprise several units of formula (III) in which at least one of the R^{R}, R⁶, R¹⁰, R¹¹, m₁, and m₂ values is different in at least one of the units.

The copolymer may also comprise at least one unit of formula (II) and at least one unit of formula (III), it being possible for the units of formula (II) and the units of formula (III) to be identical to or different from one another.

According to another embodiment, it is also possible to use a copolymer additionally comprising at least one hydrocarbon unit comprising two groups capable of establishing hydrogen interactions chosen from ester, amide, sulphonamide, carbamate, thiocarbamate, urea, urethane, thiourea, oxamido, guanidine, and biguanidino groups, and combinations thereof.

These copolymers may be chosen from block copolymers, sequential copolymers, and grafted copolymers.

Dispersion of Polymer Particles in a Liquid Fatty Phase

The composition according to the present disclosure may comprise at least one stable dispersion of essentially spherical polymer particles of at least one polymer in a physiologically acceptable liquid fatty phase.

These dispersions may be provided, for example, in the form of nanoparticles of polymers in stable dispersion in the liquid organic phase. The nanoparticles may have a mean size ranging from 5 to 800 nm, for example, from 50 to 500 nm. However, it is possible to obtain sizes for polymer particles up to 1 μm.

In at least one embodiment, the particles of polymers in dispersion may be insoluble in water-soluble alcohols, for example, ethanol.

The dispersed polymers which may be used in the composition of the present disclosure may have a molecular weight ranging from 2000 to 10 000 000 g/mol and a Tg anging from −100° C. to 300° C., for example, from −50° C. to 100° C., or from −10° C. to 50° C.

Suitable film-producing polymers may have a low Tg, i.e., less than or equal to skin temperature, for example, less than or equal to 40° C.

In at least one embodiment, the at least one film-forming polymer may be chosen from acrylic and vinyl radical homopolymers and copolymers having, for example, a Tg of less than or equal to 40° C., for instance, ranging from −10° C. to 30° C., used alone or as a blend.

As used herein, the term “radical polymer” is understood to mean a polymer obtained by polymerization of monomers possessing unsaturation, for example, ethylenic unsaturation, each monomer being capable of homopolymerizing (unlike polycondensates). The radical polymers may be vinyl polymers and copolymers, for example, acrylic polymers.

The acrylic polymers may result from the polymerization of monomers possessing ethylenic unsaturation having at least one acidic group, of the esters of these acidic monomers, and/or of the amides of these acids.

Examples of monomers carrying an acidic group include, but are not limited to, unsaturated α,β-ethylenic carboxylic acids, such as acrylic acid, methacrylic acid, crotonic acid, maleic acid, and itaconic acid. These monomers may, for example, be chosen from (meth)acrylic acid and crotonic acid. In at least one embodiment, the monomer carrying an acidic group may be (meth)acrylic acid.

The esters of acidic monomers may be chosen from esters of (meth)acrylic acid (also referred to as (meth)acrylates), such as alkyl (meth)acrylates, for example, C₁-C₂₀, or C₁-C₈, (meth)acrylates; aryl (meth)acrylates, for example, C₆-C₁₀ aryl (meth)acrylates; and hydroxyalkyl (meth)acrylates, for example, C₂-C₆ hydroxyalkyl (meth)acrylates. Non-limiting examples of alkyl (meth)acrylates include methyl, ethyl, butyl, isobutyl, 2-ethylhexyl, and lauryl (meth)acrylates. Suitable hydroxyalkyl (meth)acrylates include, for example, hydroxyethyl (meth)acrylate and 2-hydroxypropyl (meth)acrylates. Examples of aryl (meth)acrylates include, but are not limited to, benzyl and phenyl acrylate.

In at least one embodiment, the esters of (meth)acrylic acid may be chosen from alkyl (meth)acrylates.

Non-limiting examples of radical polymers include copolymers of (meth)acrylic acid and of alkyl (meth)acrylate, for example, C₁-C₄ alkyl (meth)acrylate. In one embodiment, the radical polymer may be methyl acrylate optionally copolymerized with acrylic acid.

Suitable amides of the acidic monomers include, for example, (meth)acrylamides such as N-alkyl(meth)acrylamides, for instance, N—(C₂-C₁₂ alkyl)-(meth)acrylamides, for example, N-ethylacrylamide, N-(t-butyl)acrylamide, and N-octylacrylamide, and N,N-di(C₁-C₄ alkyl)(meth)acrylamides.

The acrylic polymers may also result from the polymerization of monomers possessing ethylenic unsaturation having at least one amine group in the free form or else partially or completely neutralized form or alternatively partially or completely quaternized form. Such monomers may, for example, be chosen from dimethylaminoethyl (meth)acrylate, dimethylaminoethylmethacrylamide, vinylamine, vinylpyridine, and diallyidimethylammonium chloride.

The vinyl polymers may also result from the homopolymerization or from the copolymerization of at least one monomer chosen from vinyl esters and styrene monomers. In one embodiment of the present disclosure, these monomers may be polymerized with acidic monomers, their esters, and/or their amides, such as those mentioned above. Examples of vinyl esters include, but are not limited to vinyl acetate, vinyl propionate, vinyl neodecanoate, vinyl pivalate, vinyl benzoate,e and vinyl t-butylbenzoate. Suitable styrene monomers may be chosen, for example, from styrene and α-methylstyrene.

It is to be understood that he list of the monomers given above is not intended to be limiting in any matter and it is possible to use any monomer known to a person skilled in the art and belonging to the categories of acrylic and vinyl monomers (including monomers modified by a silicone chain).

Other examples of vinyl monomers which are suitable for use in accordance with the present disclosure include:

N-vinylpyrrolidone, vinylcaprolactam, N—(C₁-C₆ alkyl)vinylpyrroles, vinyloxazoles, vinylthiazoles, vinylpyrimidines, and vinylimidazoles, and

olefins, such as ethylene, propylene, butylene, isoprene, and butadiene.

The vinyl polymer may be crosslinked using at least one difunctional monomer comprising, for example, at least two ethylenic unsaturations, such as ethylene glycol dimethacrylate and diallyl phthalate.

The dispersed polymers of the present disclosure may be chosen, for example, from polymers or copolymers such as polyurethanes, polyurethane-acrylics, polyureas, polyurea-polyurethanes, polyester-polyurethanes, polyether-polyurethanes, polyesters, polyesteramides, alkyds; acrylic and/or vinyl polymers or copolymers; acrylicsilicone copolymers; polyacrylamides; silicone polymers such as silicone polyurethanes and acrylics; fluoropolymers, and blends thereof.

The polymer or polymers in dispersion in the fatty phase may be present, as dry matter, in an amount ranging from 5 to 40% of the weight of the composition, for example, from 5 to 35%, or from 8 to 30%.

According to one embodiment, the polymer particles in dispersion may be stabilized at the surface by at least one stabilizing agent which is solid at ambient temperature. In this case, the amount, as dry matter, of the dispersion represents the total amount of polymer+stabilizing agent, provided that the amount of polymer cannot be less than 5%.

The polymer particles may be stabilized at the surface by virtue of a stabilizing agent which may be chosen from sequential polymers, grafted polymers, and/or random polymers, and mixtures thereof. The stabilization may be carried out by any known means, for example, by direct addition of the stabilizing polymer during the polymerization.

The stabilizing agent may be present in the mixture before polymerization of the polymer. However, it is also possible to add it continuously, for instance, when the monomers are also added continuously.

The at least one stabilizing agent may be added to the starting mixture in an amount ranging from 2 to 30% by weight of stabilizing agent with respect to the starting mixture of monomers, for example, from 5 to 20% by weight.

When a grafted and/or sequential polymer is used as the stabilizing agent, the synthesis solvent may be chosen such that at least a portion of the grafts or sequences of the stabilizing polymer is soluble in the solvent, the other portion of the grafts or sequences not being soluble therein. In at least one embodiment, the stabilizing polymer used during the polymerization may be soluble, or dispersible, in the synthesis solvent. Furthermore, a stabilizing agent may be chosen such that the insoluble sequences or grafts exhibit a degree of affinity for the polymer formed during the polymerization.

Examples of suitable grafted polymers include, but are not limited to, silicone polymers grafted with a hydrocarbon chain and hydrocarbon polymers grafted with a silicone chain.

Thus, at least one stabilizing agent may be chosen from grafted or sequential block copolymers comprising at least one polyorganosiloxane block and at least one radical polymer block, such as grafted copolymers of acrylic/silicone type, which may be employed, for example, when the non-aqueous medium is a silicone medium.

The at least one stabilizing agent may also be chosen from grafted or sequential block copolymers comprising at least one polyorganosiloxane block and at least one polyether block. The polyorganosiloxane block may be chosen, for example, from polydimethylsiloxane and poly(C₂-C₁₈)alkylmethylsiloxanes; the polyether block may be a poly(C₂-C₁₈ alkylene oxide), for example, polyoxyethylene and/or polyoxypropylene. In at least one embodiment, the stabilizing agent may be chosen from dimethicone copolyols; (C₂-C₁₈)alkyl dimethicone copolyols, such as those sold under the name “Dow Corning 3225C” by Dow Corning; and lauryl methicones, such as those sold under the name “Dow Corning Q2-5200” by Dow Corning.

In another embodiment, the at least one stabilizing agent may be chosen from grafted or sequential block copolymers comprising at least one block resulting from the polymerization of at least one ethylenic monomer possessing at least one ethylenic bond which is optionally conjugated, such as ethylene and dienes, for example, butadiene and isoprene, and at least one block of a polymer chosen from vinyl and styrene polymers. When the ethylenic monomer comprises several ethylenic bonds which are optionally conjugated, the residual ethylenic unsaturations after the polymerization generally may be hydrogenated. Thus, as a known example, the polymerization of isoprene results, after hydrogenation, in the formation of an ethylene-propylene block and the polymerization of butadiene results, after hydrogenation, in the formation of an ethylene-butylene block. These polymers may include sequential copolymers, such as “diblock” and “triblock” copolymers; polystyrene/polyisoprene (SI) copolymers; polystyrene/polybutadiene (SB) copolymes, such as those sold under the name “Luvitol HSB” by BASF; polystyrene/copoly(ethylene-propylene) (SEP) copolymers, such as those sold under the name of “Kraton” by Shell Chemical Co.; and polystyrene/copoly(ethylene-butylene) (SEB) copolymers. Further suitable commercial products include, but are not limited to, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS), and Kraton D-1107 (SIS). These polymers are generally known as copolymers of hydrogenated or non-hydrogenated dienes.

Additional non-limiting examples of suitable polymers include Gelled Permethyl 99A-750, 99A-753-59, and 99A-753-58 (blend of triblock and star polymer), Versagel 5960 from Penreco (triblock+star polymer), and OS129880, OS129881, and OS84383 from Lubrizol (styrene/methacrylate copolymer).

Examples of grafted or sequential block copolymers comprising at least one block resulting from the polymerization of at least one ethylenic monomer possessing at least one ethylenic bond and at least one block of an acrylic polymer include poly(methyl methacrylate)/polyisobutylene bi- or trisequential copolymers and grafted copolymers with a poly(methyl methacrylate) backbone and with polyisobutylene grafts.

Suitable grafted or sequential block copolymers comprising at least one block resulting from the polymerization of at least one ethylenic monomer possessing at least one ethylenic bond and at least one block of a polyether, such as a poly(C₂-C₁₈ alkylene oxide) (for instance, polyoxyethylenated and/or polyoxypropylenated) may be chosen from polyoxyethylene/polybutadiene or polyoxyethylene/polyisobutylene bi- or trisequential copolymers.

When a random polymer is used as stabilizing agent, it may be chosen so that it has a sufficient amount of groups rendering it soluble in the envisaged synthesis solvent.

It is thus possible to employ copolymers based on alkyl (meth)acrylates resulting from C₁-C₄ alcohols and on alkyl (meth)acrylates resulting from C₈-C₃₀ alcohols. Suitable examples of such copolymers include stearyl methacrylate/methyl methacrylate copolymer.

When the solvent for the synthesis of the polymer is nonpolar, the stabilizing agent may be chosen from polymers which introduce the most complete covering possible of the particles, several chains of stabilizing polymers then being adsorbed on one particle of polymer obtained by polymerization.

In this case, the stabilizing agent may be chosen from grafted polymers and sequential polymers, so as to have a better interfacial activity. This improved interfacial activity may result because the sequences or grafts which are insoluble in the synthesis solvent contribute a bulkier covering to the surface of the particles.

When the synthesis solvent comprises at least one silicone oil, the stabilizing agent may be chosen from grafted or sequential block copolymers comprising at least one block of polyorganosiloxane type and at least one block of a polymer chosen from radical polymers, polyethers, and polyesters, such as polyoxypropylenated and/or polyoxyethylenated blocks.

When the synthesis solvent does not comprise a silicone oil, the stabilizing agent may be chosen from:

(a) grafted or sequential block copolymers comprising at least one block of polyorganosiloxane type and at least one block of a polymer chosen from radical polymers, polyethers, and polyesters,

(b) copolymers of alkyl (meth)acrylates resulting from C₁-C₄ alcohols and of alkyl (meth)acrylates resulting from C₈-C₃₀ alcohols, and

(c) grafted or sequential block copolymers comprising at least one block resulting from the polymerization of at least one ethylenic monomer possessing conjugated ethylenic bonds,

and at least one block of a polymer chosen from vinyl polymers, acrylic polymers, polyethers, polyesters, and mixtures thereof.

In at least one embodiment, the stabilizing agent may be chosen from diblock polymers.

Aqueous Dispersion of Polymer Particles

According to another embodiment, the at least one film-forming polymer may be chosen from aqueous dispersions of polymer particles, in the case where the composition according to the present disclosure comprises an aqueous phase.

The aqueous dispersion comprising at least one film-forming polymer may be prepared by a person skilled in the art on the basis of his overall knowledge, for example, by an emulsion polymerization or by dispersing the polymer formed beforehand.

Examples of film-forming polymers suitable for use in the aqueous dispersion include, but are not limited to, synthetic polycondensate polymers, synthetic radical polymers, polymers of natural origin, and blends thereof.

Non-limiting examples of polycondensates include anionic, cationic, non-ionic, or amphoteric polyurethanes; polyurethane-acrylics; polyurethane-polyvinylpyrrolidones; polyester-polyurethanes; polyether-polyurethanes; polyureas; polyurea-polyurethanes; and blends thereof.

The polyurethanes may, for example, be chosen from copolymers of aliphatic polyurethanes, copolymers of cycloaliphatic polyurethanes, copolymers of aromatic polyurethanes, copolymers of polyurea-polyurethanes, and copolymers of polyurea comprising, alone or as a mixture:

at least one sequence of linear or branched and aliphatic, cycloaliphatic, and/or aromatic polyester origin,

at least one sequence of aliphatic, cycloaliphatic, and/or aromatic polyether origin,

at least one substituted or unsubstituted and branched or unbranched silicone sequence, for example of polydimethylsiloxane or of polymethylphenylsiloxane, and/or

at least one sequence comprising fluorinated groups.

The polyurethanes as defined in the present disclosure may also be obtained from branched or unbranched polyesters or from alkyds comprising mobile hydrogens which are modified by means of polyaddition with a diisocyanate and a bifunctional organic coreactant compound (for example, dihydro, diamino, and hydroxy-amino), additionally comprising an entity chosen from carboxylates, carboxylic acid groups, sulphonates, sulphonic acid groups, neutralizable tertiary amine groups, and quaternary ammonium groups.

Suitable film-forming polymers may also include polyesters, polyesteramides, polyesters comprising a fatty chain, polyamides, and epoxy ester resins.

The polyesters may be obtained in a known way by means of the polycondensation of aliphatic or aromatic diacids with aliphatic diols, with aromatic diols, or with polyols. Examples of aliphatic diacids include, but are not limited to, succinic acid, glutaric acid, adipic acid, pimelic acid, suberic acid, and sebacic acid. Stuiable aromatic diacids may be chosen, for example, from terephthalic acid, isophthalic acid, and derivatives, such as phthalic anhydride. Non-limiting examples of aliphatic diols include ethylene glycol, propylene glycol, diethylene glycol, neopentyl glycol, cyclohexanedimethanol, and 4,4-N-(1-methylpropylidene)bisphenol. Examples of suitable polyols include glycerol, pentaerythritol, sorbitol, and trimethylolpropane.

The polyesteramides may be obtained in an analogous way to the polyesters, by means of the polycondensation of diacids with diamines or aminoalcohols. Non-limiting examples of diamines include ethylenediamine, hexamethylenediamine, metaphenylenediamine, and para-phenylenediamine. An example of a suitable aminoalcohol is monoethanolamine.

Suitable monomers carrying an anionic group which can be used during the polycondensation, include for example, dimethylolpropionic acid; trimellitic acid and deivatives, such as trimellitic anhydride; the sodium salt of the acid 3-sulphopentanediol; and the sodium salt of 5-sulpho-1,3-benzenedicarboxylic acid. The polyesters comprising a fatty chain may be obtained via the use, during the polycondensation, of diols comprising a fatty chain. The epoxy ester resins may be obtained by the polycondensation of fatty acids with a condensate at the α,ω-diepoxy ends.

Non-limiting examples of radical polymers include acrylic and/or vinyl polymers and copolymers. In at least one embodiment, the radical polymer may possess an anionic radical. Suitable monomers carrying an anionic group which may be used during the radical polymerization include, for example, acrylic acid, methacrylic acid, crotonic acid, maleic anhydride, and 2-acrylamido-2-methylpropanesulphonic acid.

The acrylic polymers may result from the copolymerization of monomers chosen from esters and/or amides of (meth)acrylic acid. Examples of monomers of the ester type include, but are not limited to, methyl methacrylate, ethyl methacrylate, butyl methacrylate, isobutyl methacrylate, 2-ethylhexyl methacrylate, and lauryl methacrylate. Non-limiting examples of monomers of the amide type include N-(t-butyl)acrylamide and N-(t-octyl)acrylamide.

According to one embodiment, the acrylic polymers may be obtained by the copolymerization of monomers possessing ethylenic unsaturation comprising hydrophilic groups, which, in at least one embodiment, are of nonionic nature, such as hydroxyethyl acrylate, 2-hydroxypropyl acrylate, hydroxyethyl methacrylate, and 2-hydroxypropyl methacrylate.

The vinyl polymers may result from the homopolymerization or from the copolymerization of monomers chosen from vinyl esters, styrene, and butadiene. Examples of suitable vinyl esters include, but are not limited to, vinyl acetate, vinyl neodecanoate, vinyl pivalate, vinyl benzoate, and vinyl t-butylbenzoate.

The at least one film-forming polymer may also be chosen from acrylic/silicone copolymers and nitrocellulose/acrylic copolymers.

Further examples of film-forming polymers include the polymers resulting from the radical polymerization of at least one radical monomer inside and/or partially at the surface of preexisting particles of at least one polymer chosen from polyurethanes, polyureas, polyesters, polyesteramides, and alkyds. These polymers may be generally referred to as “hybrid polymers”.

When an aqueous dispersion of polymer particles is used, the aqueous dispersion may be present, as dry matter, in an amount ranging from 3 to 60% by weight if the weight of the total composition, for example, from 10 to 50%.

The size of the polymer particles in the aqueous dispersion may range from 10 to 500 nm, for example, from 20 to 150 nm, making it possible to obtain a film having a significant gloss. However, particle sizes ranging up to one micron may also be used.

Non-limiting examples of aqueous dispersions of film-forming polymer include the acrylic dispersions sold under the names “Neocryl XK-90®”, “Neocryl A-1070®”, “Neocryl A-1090®”, “Neocryl BT-62®”, “Neocryl A-1079®”, and “Neocryl A-523®” by Avecia-Neoresins, “Dow Latex 432®” by Dow Chemical, “Daitosol 5000 AD®” and “Daitosol 5000 ®” by Daito Kasey Kogyo, and “Syntran 5760” by Interpolymer; the aqueous polyurethane dispersions sold under the names “Neorez R-981®” and “Neorez R-974®” by Avecia-Neoresins, “Avalure UR-405®”, “Avalure UR-410®”, “Avalure UR-425®”, “Avalure UR-450®”, “Sancure 875®”, “Sancure 861®”, “Sancure 878®”, and “Sancure 206®” by Goodrich, “Impranil 85®” by Bayer, and “Aquamere H-1511®” by Hydromer; the sulphopolyesters sold under the trade name “Eastman AQ®” by Eastman Chemical Products; vinyl dispersions, such as “Mexomere PAM”; aqueous dispersions of poly(vinyl acetate), such as “Vinybran® from Nisshin Chemical or those sold by Union Carbide; aqueous dispersions of vinylpyrrolidone/dimethylaminopropylmethacrylamide/lauryldimethylpropylmethacrylamidoammonium chloride terpolymer, such as Styleze W from ISP; aqueous dispersions of polyurethane/polyacrylic hybrid polymers, such as those sold under the references “Hybridur®” by Air Products and “Duromer®” from National Starch; dispersions of core/shell type for example, those sold by Atofina under the Kynar reference (core: fluorinated; shell: acrylic); those disclosed in U.S. Pat. No. 5,188,899 (core: silica; shell: silicone), and mixtures thereof.

Water-Soluble Film-Forming Polymer

In the case where the composition comprises an aqueous phase, the at least one film-forming polymer may be a water-soluble polymer. The water-soluble polymer may thus be dissolved in the aqueous phase of the composition.

Examples of water-soluble film-forming polymers include cationic polymers, such as:

(1) acrylic polymers and copolymers, such as polyacrylates and polymethacrylates; the copolymers of family (1) may additionally comprise at least one unit deriving from comonomers which may be chosen from acrylamides, methacrylamides, diacetone acrylamides, acrylamides and methacrylamides substituted on the nitrogen by lower alkyl groups, acrylic and methacrylic acids and their esters, vinyllactams, such as vinylpyrrolidone and vinylcaprolactam, and vinyl esters.

Thus, non-limiting examples of copolymers of family (1) include:

copolymers of acrylamide and of dimethylaminoethyl methacrylate quaternized with an entity chosen from dimethyl sulphate and methyl halides, such as that sold under the name Hercofloc by Hercules,

the copolymer of acrylamide and of methacryloyloxyethyltrimethylammonium chloride disclosed, for example, in European Patent Application No. 0 080 976 and sold under the name Bina Quat P 100 by Ciba-Geigy,

the copolymer of acrylamide and of methacryloyloxyethyltrimethylammonium methyl sulphate sold under the name Reten by Hercules,

vinylpyrrolidone/dialkylaminoalkyl acrylate and methacrylate copolymers, which may or may not be quaternized, such as the products sold under the name “Gafquat” by ISP, for example, “Gafquat 734” and “Gafquat 755”, and the products denoted by “Copolymer 845, 958, and 937”. These polymers are described, for example, in French Patent Nos. 2 077 143 and 2 393 573,

dimethylaminoethyl methacrylate/vinylcaprolactam/vinylpyrrolidone terpolymers, such as the product sold under the name Gaffix VC 713 by ISP, and

quaternized vinylpyrrolidone/dimethylaminopropylmethacrylamide copolymer, such as the product sold under the name “Gafquat HS 100” by ISP.

(2) quaternized polysaccharides disclosed, for instance, in U.S. Pat. Nos. 3,589,578 and 4,031,307, such as guar gums comprising trialkylammonium cationic groups. Such products are sold, for example, under the trade names Jaguar Cl3 S, Jaguar C15, and Jaguar C17 by Meyhall.

(3) quaternary copolymers of vinylpyrrolidone and of vinylimidazole;

(4) chitosans and their salts;

(5) cationic cellulose derivatives, such as the copolymers of cellulose and copolymers of cellulose derivatives grafted with a water-soluble monomer comprising a quaternary ammonium and disclosed, for example, in U.S. Pat. No. 4,131,576, such as hydroxyalkylcelluloses, for example hydroxymethyl-, hydroxyethyl-, and hydroxy-propylcelluloses, grafted, for instance, with a salt chosen from methacryloyloxyethyl-trimethylammonium, methacrylamidopropyltrimethylammonium, and dimethyldiallylammonium salts. The marketed products corresponding to this definition include, for example, the products sold under the names “Celquat L 200” and “Celquat H 100” by National Starch Company.

The water-soluble film-forming polymers may also be chosen from amphoteric polymers, for example:

1) polymers resulting from the copolymerization of a monomer derived from a vinyl compound carrying a carboxyl group, such as acrylic acid, methacrylic acid, maleic acid, and x-chloroacrylic acid, and of a basic monomer derived from a substituted vinyl compound comprising at least one basic atom, such as dialkylaminoalkyl methacrylates and acrylates and dialkylaminoalkylmethacrylamides and -acrylamides. Such compounds are disclosed, for example, in U.S. Pat. No. 3,836,537.

2) polymers comprising units deriving:

a) from at least one monomer chosen from acrylamides and methacrylamides substituted on the nitrogen atom by an alkyl group,

b) from at least one acidic comonomer comprising at least one reactive carboxyl group, and

c) from at least one basic comonomer, such as esters, having primary, secondary, tertiary, and quaternary amine substituents, of acrylic and methacrylic acids and the quaternization product of dimethylaminoethyl methacrylate with dimethyl or diethyl sulphate.

(3) crosslinked alkylpolyaminoamides derived completely or partially from polyaminoamides.

(4) polymers comprising zwitterionic units.

(5) polymers derived from chitosan.

(6) polymers derived from the N-carboxyalkylation of chitosan, such as the N-(carboxymethyl)chitosan and the N-(carboxybutyl)chitosan sold under the name “Evalsan” by Jan Dekker.

(7) (C₁-C₅)alkyl vinyl ether/maleic anhydride copolymers partially modified by semiamidation with an N,N-dialkylaminoalkylamine, such as N,N-dimethylaminopropylamine, or by semiesterification with an N,N-dialkanolamine. These copolymers may also comprise other vinyl comonomers, such as vinylcaprolactam.

In one embodiment of the present disclosure, the water-soluble film-forming polymers may be chosen from:

proteins, such as proteins of vegetable origin, for example, wheat and soybean proteins;

proteins of animal origin, such as keratin, for example, keratin hydrolysates and sulphonic keratins;

anionic, cationic, amphoteric, or nonionic polymers of chitin;

anionic, cationic, amphoteric, or nonionic polymers of chitosan;

cellulose polymers, such as hydroxyethylcellulose, hydroxypropylcellulose, methylcellulose, ethylhydroxyethylcellulose, carboxymethylcellulose, and quaternized cellulose derivatives;

acrylic polymers and copolymers, such as polyacrylates and polymethacrylates;

vinyl polymers, such as polyvinylpyrrolidones, copolymers of methyl vinyl ether and of maleic anhydride, the copolymer of vinyl acetate and of crotonic acid, and copolymers of vinylpyrrolidone and of vinyl acetate;

copolymers of vinylpyrrolidone and of caprolactam; poly(vinyl alcohol)s;

optionally modified polymers of natural origin, such as:
gum arabic, guar gum, xanthan derivatives, and karaya gum;
alginates and carrageenans;
glycoaminoglycans, hyaluronic acid, and its derivatives;
shellac, sandarac gum, dammars, elemis, and copals;
deoxyribonucleic acid; and
mucopolysaccharides, such as hyaluronic acid, and chondroitin sulphate, and

mixtures thereof.

These polymers may be used, for instance, if fairly significant removal of the film with water is desired.

Polymers having a Nonsilicone Organic Backbone Grafted with Monomers Comprising a Polysiloxane

According to one embodiment of the present disclosure, the at least one film-forming polymer may be chosen from polymers having a nonsilicone organic backbone grafted with monomers comprising a polysiloxane. These polymers may be chosen from fat-soluble, fat-dispersible, and water-soluble polymers and polymers which are dispersible in an aqueous medium.

The polymers having a nonsilicone organic backbone grafted with monomers comprising a polysiloxane comprise a main organic chain formed of organic monomers not comprising silicone to which at least one polysiloxane macromer is grafted, within the main chain and, optionally, on at least one of the ends of the latter.

As used herein, the expression “polysiloxane macromer” denotes, as is generally accepted, any monomer comprising a polymer chain of the polysiloxane type in its structure.

The nonsilicone organic monomers constituting the main chain of the grafted silicone polymer may be chosen from monomers possessing ethylenic unsaturation which can be polymerized by the radical method, monomers which can be polymerized by polycondensation, such as those forming polyamides, polyesters, and polyurethanes, and monomers with an opening ring, such as those of the oxazoline and caprolactone type.

The polymers having a nonsilicone organic backbone grafted with monomers comprising a polysiloxane according to the present disclosure may be obtained in accordance with any method known to a person skilled in the art, for example, by the reaction between (i) a starting polysiloxane macromer correctly functionalized on the polysiloxane chain and (ii) at least one nonsilicone organic compound, itself correctly functionalized by a functional group which is capable of reacting with the functional group or groups carried by the said silicone with the formation of a covalent bond; a non-limiting example of such a reaction is the radical reaction between a vinyl group carried at one of the ends of the silicone and a double bond of a monomer possessing ethylenic unsaturation of the main chain.

The polymers having a nonsilicone organic backbone grafted with monomers comprising a polysiloxane according to the present disclosure may be chosen from those disclosed in U.S. Pat. Nos. 4,693,935, 4,728,571, and 4,972,037, European Patent Nos. 0 412 704, 0 412 707, and 0 640 105, and International Patent Application Publication No. WO 95/00578. Described are copolymers obtained by radical polymerization starting from monomers possessing ethylenic unsaturation and from monomers having a vinyl end group or else copolymers obtained by the reaction of a polyolefin comprising functionalized groups and a polysiloxane macromer having an end functional group which reacts with the said functionalized groups.

Other non-limiting of grafted silicone polymers suitable for use in accordance with the present disclosure include the grafted silicone polymers comprising:

a) from 0 to 98% by weight of at least one lipophilic monomer (A) of low polarity possessing ethylenic unsaturation which may be polymerized by the radical method;

b) from 0 to 98% by weight of at least one hydrophilic polar monomer (B) possessing ethylenic unsaturation which may be copolymerized with the at least one monomer of type (A); and

c) from 0.01 to 50% by weight of at least one polysiloxane macromer (C) of general formula (IV):
X(Y)ₙSi(R)₃₋ₘZₘ (IV)

in which:

X is a vinyl group which may be copolymerized with the monomers (A) and (B);

Y is a group having a divalent bond;

R is chosen from hydrogen, C₁-C₆ alkyl groups, C₁-C₆ alkoxy groups, and C₆-C₁₂ aryl groups;

Z is a monovalent polysiloxane unit having a number-average molecular weight of at least 500;

n is an integer ranging from 0 to 1; and

m is an integer ranging from 1 to 3;

the percentages being calculated with respect to the total weight of the monomers (A), (B) and (C).

These polymers may have a number-average molecular weight ranging from 10 000 to 2 000 000 and a glass transition temperature Tg or a crystalline melting point M.p. of at least −20° C.

Examples of lipophilic monomers (A) include, but are not limited to, esters of C₁-C₁₈ alcohols and of (meth)acrylic acid; esters of C₁₂-C₃₀ alcohols and of methacrylic acid; styrene; polystyrene macromers; vinyl acetate; vinyl propionate; α-methylstyrene; tertbutylstyrene; butadiene; cyclohexadiene; ethylene; propylene; vinyltoluene; esters of (meth)acrylic acid and of 1,1-dihydroperfluoroalkanols or of homologues of the latter; esters of (meth)acrylic acid and of ω-hydrofluoroalkanols; esters of (meth)acrylic acid and of fluoroalkylsulphonamido alcohols; esters of (meth)acrylic acid and of fluoroalkyl alcohols; esters of (meth)acrylic acid and of alcohol fluoroethers; and mixtures thereof. In at least one embodiment, the at least one monomer (A) may be chosen from n-butyl methacrylate, isobutyl methacrylate, tert-butyl acrylate, tert-butyl methacrylate, 2-ethylhexyl methacrylate, methyl methacrylate, 2-(N-methylperfluorooctanesulphonamido)ethyl acrylate, 2-(N-butylperfluorooctanesulphonamido)ethyl acrylate, and mixtures thereof.

Non-limiting examples of polar monomers (B) include acrylic acid, methacrylic acid, N,N-dimethylacrylamide, dimethylaminoethyl methacrylate, quaternized dimethylaminoethyl methacrylate, (meth)acrylamide, N-(t-butyl)acrylamide, maleic acid, maleic anhydride, and the hemiesters of these, hydroxyalkyl (meth)acrylates, diallyldimethylammonium chloride, vinylpyrrolidone, vinyl ethers, maleimides, vinylpyridine, vinylimidazole, vinyl and heterocyclic polar compounds, styrenesulphonate, allyl alcohol, vinyl alcohol, vinylcaprolactam, and mixtures thereof. In at least one embodiment, the at least one monomer (B) may be chosen from acrylic acid, N,N-dimethylacrylamide, dimethylaminoethyl methacrylate, quaternized dimethylaminoethyl methacrylate, vinylpyrrolidone, and mixtures thereof these.

Suitable exemplary commercial products include the products KP 561 and KP 562 sold by Shin-Etsu such that the monomer (A) is chosen from esters of C₁₈-C₂₂ alcohols and of methacrylic acid.

The at least one polysiloxane macromer (C) of formula (IV) may be chosen from those corresponding to the following general formula (V):

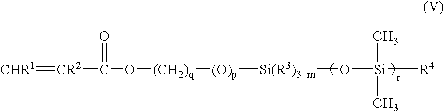

in which:

R¹ is chosen from hydrogen and —COOH (preferably hydrogen);

R² is chosen from hydrogen, methyl, and —CH₂COOH (preferably methyl);

R³ is chosen from C₁-C₆ alkyl, alkoxy, and alkylamino groups, C₆-C₁₂ aryl groups, and hydroxyl (preferably methyl);

R⁴ is chosen from C₁-C₆ alkyl, alkoxy, and alkylamino groups, C₆-C₁₂ aryl groups, and hydroxyl (preferably methyl);

q is an integer ranging from 2 to 6 (preferably 3);

p is an integer ranging from 0 or 1;

r is an integer ranging from 5 to 700; and

m is an integer ranging from 1 to 3 (preferably 1).

In one embodiment, in formula (V),

R¹ may be hydrogen;

R² may be a methyl group;

R³ may be a methyl group;

R⁴ may be a methyl group;

q may be equal to 3; and

m may be equal to 1).

In another embodiment, the at least one polysiloxanes macromonomer C may be chosen from polysiloxane macromers of formula (VI):

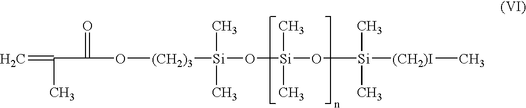

wherein n is a number ranging from 5 to 700 and I is an integer ranging from 0 to 3.

One embodiment of the present disclosure comprises the use of a copolymer capable of being obtained by radical polymerization starting from a mixture of monomers comprising:

a) 60% by weight of tert-butyl acrylate;

b) 20% by weight of acrylic acid; and

c) 20% by weight of silicone macromer of formula (VII):

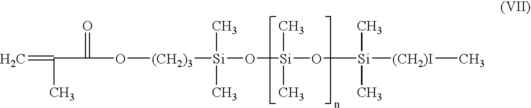

wherein n is a number ranging from 5 to 700 and I is an integer ranging from 0 to 3, the percentages by weight being calculated with respect to the total weight of the monomers.

Another embodiment of the present disclosure comprises the use of a copolymer capable of being obtained by radical polymerization starting from a mixture of monomers comprising:

a) 80% by weight of tert-butyl acrylate; and

b) 20% by weight of silicone macromer of formula (VI II):

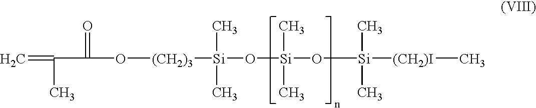

wherein n is a number ranging from 5 to 700 and I is an integer ranging from 0 to 3, the percentages by weight being calculated with respect to the total weight of the monomers.

Other grafted silicone polymers having a nonsilicone organic backbone suitable for use in accordance with the present disclosure include the grafted silicone copolymers capable of being obtained by the reactive extrusion of a polysiloxane macromer possessing an end functional group which reacts with a polymer of the polyolefin type comprising reactive groups capable of reacting with the end functional group of the polysiloxane macromer in order to form a covalent bond which enables the silicone to be grafted to the main chain of the polyolefin. These polymers, and the process for the preparation thereof, are disclosed, for example, in International Patent Application Publication No. WO 95/00578.

The reactive polyolefins may be chosen from polyethylenes and polymers of monomers derived from ethylene, such as propylene, styrene, alkylstyrene, butylene, butadiene, (meth)acrylates, vinyl esters, and equivalents, comprising reactive functional groups capable of reacting with the end functional group of the polysiloxane macromer. In at least one embodiment, the reactive polyolefins may be chosen from copolymers of ethylene, and copolymers of ethylene derivatives and of monomers chosen from those comprising a carboxyl functional group, such as (meth)acrylic acid; those comprising an acid anhydride functional group, such as the anhydride of maleic acid; those comprising an acid chloride functional group, such as the chloride of (meth)acrylic acid; those comprising an ester functional group, such as the esters of (meth)acrylic acid; and those comprising an isocyanate functional group.

The silicone macromers may be chosen from polysiloxanes comprising a functionalized group, at the end of the polysiloxane chain or close to the end of the chain, chosen from alcohols, thiols, epoxy groups, and primary and secondary amines chosen, for example, from those corresponding to the general formula (IX):
T-(CH₂)ₛ—Si—[—(OSiR⁵R⁶)ₜ—R⁷]_{y} (IX)

in which T is chosen from NH₂, NHRN, epoxy functional groups, OH, and SH; R⁵, R⁶, R⁷, and RN are independently chosen from hydrogen and C₁-C₆ alkyl, phenyl, benzyl, and C₆-C₁₂ alkylphenyl groups; s is a number ranging from 2 to 100; t is a number ranging from 0 to 1000 and y is a number ranging from 1 to 3. The silicone macromers may have a number-average molecular weight ranging from 5000 to 300 000, for example, from 8000 to 200 000, or from 9000 to 40 000.

According to one embodiment, the at least one film-forming polymer may be purchased from Minnesota Mining and Manufacturing Company under the trade names of “Silicone Plus” polymers. For example, poly(isobutyl methacrylate-co-methyl-FOSEA)-g-poly(dimethylsiloxane) is sold under the trade name SA 70-5 IBMMF.

According to another embodiment of the present disclosure, the at least one film-forming polymer may be chosen from silicone polymers grafted with nonsilicone organic monomers. These polymers may be chosen from fat-soluble, fat-dispersible,and water-soluble polymers and polymers which are dispersible in an aqueous medium.

The grafted silicone polymer or polymers having a polysiloxane backbone grafted with nonsilicone organic monomers may comprise a main silicone (or polysiloxane (/SiO—)ₙ) chain on which is grafted, within the said chain and optionally at at least one of its ends, at least one organic group not comprising silicone.

The polymers having a polysiloxane backbone grafted with nonsilicone organic monomers according to the present disclosure may be chosen from existing commercial products or alternatively they may be obtained by any means known to a person skilled in the art, for example, by a reaction between (i) a starting silicone correctly functionalized on at least one of these silicon atoms and (ii) a nonsilicone organic compound itself correctly functionalized with a functional group which is capable of reacting with the at least one functional group carried by the silicone with the formation of a covalent bond; non-limiting examples of such a reaction is the hydrosilylation reaction between /Si—H groups and CH₂═CH— vinyl groups and the reaction between —SH thio-functional groups and these same vinyl groups.

Examples of polymers having a polysiloxane backbone grafted with nonsilicone organic monomers suitable for use in the present disclosure, as well as methods of preparation, are disclosed, for example, in European Patent No. 0 582 152 and International Patent Application Publication Nos. WO 93/23009 and WO 95/03776, the teachings of which are incorporated herein by reference.

According to one embodiment of the present disclosure, the silicone polymer having a polysiloxane backbone grafted with nonsilicone organic monomers comprises the result of a radical copolymerization between at least one anionic nonsilicone organic monomer possessing ethylenic unsaturation and/or one hydrophobic nonsilicone organic monomer possessing ethylenic unsaturation and, or alternatively, a silicone exhibiting, in its chain, at least one functional group, and in one embodiment, several functional groups, capable of reacting with the said ethylenic unsaturations of the said nonsilicone monomers with the formation of a covalent bond, for example, thio-functional groups.

According to the present disclosure, the anionic monomers possessing ethylenic unsaturation may be chosen from linear or branched unsaturated carboxylic acids, optionally partially or completely neutralized in the form of a salt, and mixtures thereof, these unsaturated carboxylic acids being chosen, for example, from acrylic acid, methacrylic acid, maleic acid, itaconic acid, fumaric acid, and crotonic acid. Suitable salts may include, for example, alkali metal, alkaline earth metal, and ammonium salts. It should be noted that, likewise, in the final grafted silicone polymer, the organic group of anionic nature which comprises the result of the radical (homo)polymerization of at least one anionic monomer of unsaturated carboxylic acid type may, after reaction, be post-neutralized with a base (sodium hydroxide, ammonia, and the like) in order to convert it to the form of a salt.

According to the present disclosure, the hydrophobic monomers possessing ethylenic unsaturation may be chosen from alkanol acrylic acid esters and/or alkanol methacrylic acid esters, and mixtures thereof. The alkanols may be C₁-C₃₀ alkanols, for example, C₁-C₂₂ alkanols. In at least one embodiment, the hydrophobic monomers may be chosen from isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate, and mixtures thereof.

Examples of silicone polymers having a polysiloxane backbone grafted with nonsilicone organic monomers suitable for use in accordance with the present disclosure include silicone polymers comprising, in their structure, the following unit of formula (X):

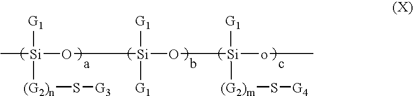

in which the G₁ radicals, which may be identical or different, are chosen from hydrogen, C₁-C₁₀ alkyl radicals, and phenyl; the G₂ radicals, which may be identical or different, are chosen from C₁-C₁₀ alkylene groups; G₃ is a polymer residue resulting from the (homo)polymerization of at least one anionic monomer possessing ethylenic unsaturation; G₄ is a polymer residue resulting from the (homo)polymerization of at least one hydrophobic monomer possessing ethylenic unsaturation; m and n are equal to 0 or 1; a is an integer ranging from 0 to 50; b is an integer ranging from 10 and 350 and c is an integer ranging from 0 to 50, with the proviso that one of the parameters a and c is not equal to 0.

The unit of formula (X) of the above text may have at least one of the following characteristics:

the G₁ radicals may denote an alkyl radical, for example, a methyl radical;

n does not equal zero and the G₂ radicals are divalent C₁-C₃ radicals, for example, propylene radicals;

G₃ is a polymer radical resulting from the (homo)polymerization of at least one monomer of the carboxylic acid possessing ethylenic unsaturation type, for example, acrylic acid and/or methacrylic acid; and

G₄ is a polymer radical resulting from the (homo)polymerization of at least one monomer of the (C₁-C₁₀)alkyl (meth)acrylate type, for example, isobutyl and methyl (meth)acrylate.

Examples of silicone polymers corresponding to the formula (X) include polydimethylsiloxanes (PDMSs) on which are grafted, via a subsidiary link of the thiopropylene type, mixed polymer units of the poly((meth)acrylic acid) type and of the poly(alkyl (meth)acrylate) type.

Other examples of silicone polymers corresponding to the formula (X) include polydimethylsiloxanes (PDMSs) on which are grafted, via a subsidiary link of the thiopropylene type, polymer units of the poly(isobutyl (meth)acrylate) type.

Such polymers may include polymers comprising at least one group of formula (XI):

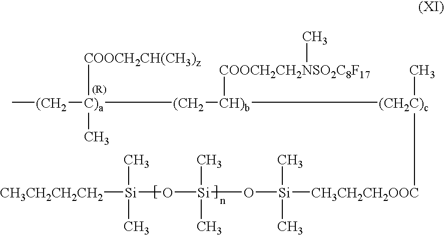

in which:

a, b, and c, which may be identical or different, are numbers ranging from 1 to 100 000, and the end groups, which may be identical or different, are each chosen from linear C₁-C₂₀ alkyl groups, branched-chain C₃-C₂₀ alkyl groups, C₃-C₂₀ aryl groups, linear C₁-C₂₀ alkoxy groups, and branched C₃-C₂₀ alkoxy groups.

Such polymers are disclosed, for example, in U.S. Pat. Nos. 4,972,037, 5,061,481, 5,209,924, 5,849,275, and 6,033,650 and International Patent Application Publication Nos. WO 93/23446 and WO 95/06078.

Additional examples of silicone polymers having a polysiloxane backbone grafted with nonsilicone organic monomers which are suitable for use in the present disclosure include silicone polymers comprising, in their structure, the following unit of formula (XII):

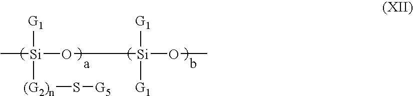

in which the G₁ and G₂ radicals have the same meaning as above in formula (X); G₅ is a polymer residue resulting from the (homo)polymerization of at least one hydrophobic monomer possessing ethylenic unsaturation or from the copolymerization of at least one anionic monomer possessing ethylenic unsaturation and of at least one hydrophobic monomer possessing ethylenic unsaturation; n is equal to 0 or 1; a is an integer ranging from 0 to 50; and b is an integer ranging from 10 to 350; with the proviso that a is not equal to 0.

The unit of formula (XII) above may have at least one of the following characteristics:

the G₁ radicals are alkyl radicals, for example, methyl radicals; and

n is not equal to zero and the G₂ radicals are chosen from divalent C₁-C₃ radicals, for example, propylene radicals.

The number-average molecular mass of the silicone polymers having a polysiloxane backbone grafted with nonsilicone organic monomers of the present disclosure may range from 10 000 to 1 000 000, for example, from 10 000 to 100 000.

The at least one film-forming agent may be present in the composition in an amount ranging from 0.5 to 60% by weight, for example, from 1 to 40%, or from 2 to 30% by weight of dry matter, with respect to the total weight of the composition.

More generally, the at least one film-forming polymer should be present in an amount sufficient to form, on the skin and/or lips, a cohesive film capable of following the movements of the skin and/or lips without becoming detached or splitting.

When the polymer has a glass transition temperature which is too high for the use desired, a plasticizer may be combined therein so as to lower this temperature of the mixture used. The plasticizer may be chosen from the plasticizers commonly used in the field of application, for instance, from compounds which can be solvents for the polymer.

Pasty Substance

The composition of the present disclosure may further comprise at least one pasty compound.

In at least one embodiment the composition disclosed herein may be devoid of lanolin or of lanolin derivatives.

Examples of lanolin derivatives include, but are not limited to, liquid lanolin, reduced lanolin, lanolin purified by adsorption, acetylated lanolin, oxypropylenated (5 PO) lanolin wax, liquid lanolin acetate, hydroxylanolin, polyoxyethylene lanolin, lanolin fatty acid, hard lanolin fatty acid, cholesteryl esters of lanolin fatty acid, lanolin alcohol, acetylated lanolin alcohol, and isopropyl lanolate.

Lanolins exhibit the disadvantage of being sensitive to heat and to ultraviolet radiation. They also may have a tendency to oxidize with release of an unpleasant smell and their very yellow color prevents them from being used in unpigmented care bases and colorless bases, which limits their use in cosmetic compositions.

The present inventors have discovered that the alkoxylated esters described above are good substitutes for lanolin and for its derivatives.

As used herein, the term “pasty substance” is intended to denote a lipophilic fatty compound, with a reversible solid/liquid change in state, which comprises a liquid fraction and a solid fraction at a temperature of 23° C. The term “pasty substance” may also be understood to mean poly(vinyl laurate).

The pasty compound is advantageously chosen from:

lanolin and its derivatives,

polymeric or nonpolymeric fluorinated compounds,

polymeric or nonpolymeric silicone compounds,

vinyl polymers, such as:
olefin homopolymers,
olefin copolymers,
hydrogenated diene homopolymers and copolymers,
linear or branched oligomers which are homo- or copolymers of alkyl (meth)acrylates comprising, for example, a C₈-C₃₀ alkyl group,
oligomers which are homo- and copolymers of vinyl esters comprising C₈-C₃₀ alkyl groups,
oligomers which are homo- and copolymers of vinyl ethers comprising C₈-C₃₀ alkyl groups,
fat-soluble polyethers resulting from polyetherification of at least one C₂-C₁₀₀ diol, for example, C₂-C₅₀ diols,
esters, and
mixtures thereof.

Non-limiting examples of fat-soluble polyethers include copolymers of ethylene oxide and/or of propylene oxide with alkylene oxides possessing a long C₆-C₃₀ chain, for example, such that the ratio by weight of the ethylene oxide and/or propylene oxide to alkylene oxides in the copolymer ranges from 5:95 to 70:30. In one embodiment, the fat-soluble polyether may include copolymers such that the long-chain alkylene oxides are arranged in blocks having an average molecular weight of 1000 to 10 000, for example a polyoxyethylene/polydodecyl glycol block copolymer, such as the ethers of dodecanediol (22 mol) and of polyethylene glycol (45 EO) sold by Akzo Nobel under the trade name Elfacos ST9.

Other suitable pasty esters may include, for example:

esters of an oligomeric glycerol, for example, esters of diglycerol, such as condensates of adipic acid and of glycerol, for which a portion of the hydroxyl groups of the glycerols have reacted with a mixture of fatty acids, such as stearic acid, capric acid, isostearic acid, and 12-hydroxystearic acid, for instance, those sold under the trade name Softisan 649 by Sasol,

arachidyl propionate, sold under the trade name Waxenol 801 by Alzo,

phytosterol esters,

noncrosslinked polyesters resulting from the polycondensation between a linear or branched C₄-C₅₀ di- or polycarboxylic acid and a C₂-C₅₀ diol or polyol, other than the polyester described above, and

ester aliphatic esters resulting from the esterification of an aliphatic hydroxycarboxylic acid ester with an aliphatic monocarboxylic acid, and their mixtures, such as
the ester resulting from the esterification reaction of hydrogenated castor oil with isostearic acid in the proportions of 1 to 1 (1/1) or hydrogenated castor oil monoisostearate,
the ester resulting from the esterification reaction of hydrogenated castor oil with isostearic acid in the proportions of 1 to 2 (1/2) or hydrogenated castor oil diisostearate,
the ester resulting from the esterification reaction of hydrogenated castor oil with isostearic acid in the proportions of 1 to 3 (1/3) or hydrogenated castor oil triisostearate, and
mixtures thereof.

In at least one embodiment, the pasty compounds may be chosen from those of vegetable origin, mixtures of soybean sterols, and oxyethylenated (5 EO) oxypropylenated (5 PO) pentaerythritol sold under the reference Lanolide by Vevy.

The pasty compound may be present in the composition in an amount ranging from 1 to 99%, for example, from 1 to 60%, from 2 to 30%, or from 5 to 15% by weight of the composition.

Coloring Materials

The composition of the present disclosure may further comprise at least one coloring material which may be chosen from dyes, pigments, pearlescent agents, and mixtures thereof. This coloring material may be present in the composition in an amount ranging from 0.001 to 98%, for example, from 0.5 to 85%, or from 1 to 60% by weight, relative to the total weight of the composition.

The dyes may be fat-soluble dyes, although water-soluble dyes may also be used. Suitable fat-soluble dyes include, for example, Sudan red, D & C Red 17, D & C Green 6,β-carotene, soybean oil, Sudan brown, D & C Yellow 11, D & C Violet 2, D & C Orange 5, quinoline yellow, and annatto. The fat-soluble dyes may be present in the composition in an amount ranging from 0 to 20% by weight, relative to the weight of the composition, for example from 0.1 to 6% by weight. The water-soluble dyes may be chosen, for example, from beetroot juice and methylene blue and, if present, may be present in the composition in an amount ranging from 0.1 to 6% by weight relative to the total weight of the composition.

For a composition in a paste or cast form, such as lipsticks or products for making up the body, the coloring material may be present in the composition in an amount ranging from 0.5 to 50%, for example, from 2 to 40%, or from 5 to 30%, with respect to the total weight of the composition.

As used herein, the term “pigments” should be understood as meaning white or colored and inorganic or organic particles which are insoluble in the liquid fatty phase and which are intended to color and/or to opacify the composition. As used herein, the term “fillers” should be understood as meaning colorless or white, inorganic or synthetic and lamellar or nonlamellar particles. As used herein, the term “pearlescent agents” should be understood as meaning iridescent particles, for example, those produced by certain shellfish in their shells and synthesized irridescent particles. These fillers and pearlescent agents may be used to modify the texture of the composition.

The pigments may be present in the composition in an amount ranging from 0.05 to 30% by weight, relative to the total weight of the final composition, for example, from 2 to 20%. Examples of inorganic pigments which can be used in the present disclosure, but are not limited to, titanium, zirconium, cerium, zinc, iron, and chromium oxides and ferric blue. Organic pigments suitable for use in the present disclosure include carbon black and barium, strontium, calcium (D & C Red No. 7), and aluminium lakes.

The pearlescent agents may be present in the composition in an amount ranging from 0.001 to 20% by weight, relative to the total weight of the composition, for example, from 1 to 15%. Non-limiting examples of pearlescent agents which may be used in the present disclosure include mica covered with titanium oxide, mica covered with iron oxide, mica covered with natural pigment, and mica covered with bismuth oxychloride, such as colored titanium oxide-coated mica.

The composition may comprise goniochromatic pigments, for example, interferential multilayer pigments, and/or reflecting pigments. These two types of pigments are disclosed, for example, in French Patent Application No. 0 209 246, which is incorporated herein by reference in its entirety.

Fillers

The composition may also comprise at least one filler which may be present in the composition in an amount ranging from 0.001 to 35% by weight, relative to the total weight of the composition, for example, from 0.5 to 15%.

Examples of suitable fillers include, but are not limited to:

talc, mica, kaolin, and starch,

Nylon® powders (in particular Orgasol),

polyethylene powders,

polytetrafluoroethylene (Teflon®) powders,

boron nitride,

microspheres formed of copolymers, such as Expancel® (Nobel Industrie),

Polytrap® 603 (Dow Corning),

Polypore® L 200 (Chemdal Corporation),

silicone resin microbeads (Tospearl® from Toshiba, for example),

silica-based fillers, such as Aerosil 200 and Aerosil 300; Sunsphere L-31 and Sunsphere H-31, sold by Asahi Glass; Chemicelen, sold by Asahi Chemical; and composites of silica and titanium dioxide, such as the TSG series sold by Nippon Sheet Glass, and

polyurethane powders, for example, powders formed of crosslinked polyurethane comprising a copolymer, said copolymer comprising trimethylol hexyllactone. For example, a hexamethylene diisocyanate/trimethylol hexyllactone polymer may be used. Such particles are available commercially, for example, under the name of Plastic Powder D-400® and Plastic Powder D-800® from Toshiki.

The filler may, for example, be a filler having a mean particle size of less than 100 μm, for example, ranging from 1 to 50 μm, or from 4 to 20 μm.

The filler may have any shape, for example, essentially spherical or in the form of platelets.

Wax

The composition may additionally comprise at least one wax. As used herein, the term “wax”, within the meaning of the present disclosure, is understood to mean a lipophilic fatty compound which is solid at ambient temperature (25° C.), which exhibits a reversible solid/liquid change in state, which has a melting point of greater than 30° C. and which can range up to 200° C., which has a hardness of greater than 0.5 MPa, and which exhibits, in the solid state, an anisotropic crystalline arrangement. On bringing the wax to its melting point, it is possible to render it miscible with oils and to form a microscopically homogeneous mixture, but, on bringing the temperature of the mixture back to ambient temperature, recrystallization of the wax in the oils of the mixture is obtained.

The waxes which may be used in the present disclosure include compounds which are solid at ambient temperature and which are intended to structure the composition, in particular in the stick form; they may be chosen, for example, from hydrocarbon, fluorinated, and/or silicone waxes and can be of plant, mineral, animal, and/or synthetic origin. In at least one embodiment, they may exhibit a melting point of greater than 40° C., for example, greater than 45° C.

Examples of waxes which may be used in accordance with the present disclosure include those generally used in the cosmetics field, for example, waxes of natural origin, such as beeswax, carnauba wax, candelilla wax, ouricury wax, Japan wax, cork fiber wax, sugarcane wax, rice wax, montan wax, paraffin wax, lignite wax, microcrystalline wax, ceresin, ozokerite, and hydrogenated oils, such as jojoba oil; synthetic waxes, such as the polyethylene waxes resulting from the polymerization or copolymerization of ethylene and Fischer-Tropsch waxes and fatty acid esters, such as octacosanyl stearate and glycerides, which are solid at 40° C. or at 45° C.; silicone waxes, such as alkyl and alkoxy dimethicones having an alkyl or alkoxy chain comprising from 10 to 45 carbon atoms and poly(di)methylsiloxane esters which are solid at 40° C., the ester chain of which comprises at least 10 carbon atoms; and mixtures thereof.

The composition according to the present disclosure may comprise polyethylene wax with a weight-average molecular mass ranging from 300 to 700, for example, equal to 500 g/mol.

The at least one wax may be represent in the composition in an amount ranging from 0.01 to 50%, for example, from 2 to 40%, or from 5 to 30% by weight, relative to the total weight of the composition.

Nonvolatile Oil

The composition may also comprise at least one nonvolatile oil other than the ester of an alkoxylated alcohol. The nonvolatile oil may be chosen from:

hydrocarbon oils of animal origin, such as perhydrosqualene;

hydrocarbon vegetable oils, such as liquid triglycerides of fatty acids comprising from 4 to 10 carbon atoms, for example, triglycerides of heptanoic acid, octanoic acid, and jojoba oil;

linear or branched hydrocarbons of mineral or synthetic origin, such as liquid paraffins and their derivatives and liquid petrolatum;

hydrocarbon esters of formula RCOOR′ in which RCOO represents a carboxylic acid residue comprising from 2 to 30 carbon atoms and R′ represents a hydrocarbon chain comprising from 1 to 30 carbon atoms, such as isononyl isononanoate, oleyl erucate, and 2-octyidodecyl neopentanoate;

fatty alcohols comprising from 12 to 26 carbon atoms, such as octyidodecanol, 2-butyloctanol, 2-hexyldecanol, 2-undecylpentadecanol,and oleyl alcohol;

fluorinated oils optionally partially comprising hydrocarbon and/or silicone components;

silicone oils, such as volatile or nonvolatile and linear or cyclic polydimethylsiloxanes (PDMSs); polydimethylsiloxanes comprising pendent alkyl, alkoxy, and/or phenyl groups or alkyl, alkoxy, and/or phenyl groups at the end of the silicone chain, which groups comprise from 2 to 24 carbon atoms; and phenylated silicones, such as phenyl trimethicones (such as the phenyl trimethicone sold under the trade name DC556 by Dow Corning), phenyl dimethicones, phenyl(trimethylsiloxy)diphenylsiloxanes, diphenyl dimethicones, diphenyl(methyldiphenyl)trisiloxanes, and (2-phenylethyl)trimethylsiloxy-silicates;

fatty acids comprising from 12 to 26 carbon atoms, such as oleic acid;

hydroxylated oils; and

mixtures thereof.

Nonvolatile Oil of High Molecular Mass

According to one embodiment, the composition may comprise a nonvolatile oil of high molecular mass, for example, ranging from 650 to 10 000 g/mol.

The composition according to the present disclosure may comprise at least one oil with a molar mass ranging from 650 to 10 000 g/mol, for example, from 900 to 7500 g/mol. This oil may be present in the composition in an amount ranging from 2 to 30%, for example, from 5 to 25%, or from 5 to 15%, by weight relative to the total weight of the composition.

Oils with a molecular mass ranging from 650 to 10 000 g/mol may be chosen from:

polybutylenes, such as Indopol H-100 (with a molar mass or MM of 965 g/mol), Indopol H-300 (MM=1340 g/mol), and Indopol H-1500 (MM=2160 g/mol), sold or manufactured by Amoco,

hydrogenated polyisobutylenes, such as Panalane H-300 E, sold or manufactured by Amoco (MM=1340 g/mol), Viseal 20000, sold or manufactured by Synteal (MM=6000 g/mol), and Rewopal PIB 1000, sold or manufactured by Witco (MM=1000 g/mol),

polydecenes and hydrogenated polydecenes, such as Puresyn 150 (MM=9200 g/mol), sold by Mobil Chemicals,

vinylpyrrolidone copolymers, such as the vinylpyrrolidone/1-hexadecene copolymer, Antaron V-216, sold or manufactured by ISP (MM=7300 g/mol), and

esters, such as:
a) esters of linear fatty acids having a total carbon number ranging from 35 to 70, such as pentaerythrityl tetrapelargonate (MM=697.05 g/mol),
b) hydroxylated esters, such as polyglyceryl-2 triisostearate (MM=965.58 g/mol),
c) aromatic esters, such as tridecyl trimellitate (MM=757.19 g/mol),
d) esters of branched C₂₄-C₂₈ fatty acids or fatty alcohols, such as those disclosed in European Patent Application No.0 955 039, for example, triisoarachidyl citrate (MM=1033.76 g/mol), pentaerythrityl tetraisononanoate (MM=697.05 g/mol), glyceryl triisostearate (MM=891.51 g/mol), glyceryl tri(2-decyltetradecanoate) (MM=1143.98 g/mol), pentaerythrityl tetraisostearate (MM=1202.02 g/mol), polyglyceryl-2 tetraisostearate (MM=1232.04 g/mol), and pentaerythrityl tetra(2-decyltetradecanoate) (MM=1538.66 g/mol), and
e) dimer diol esters and polyesters, such as esters of a dimer diol and of a fatty acid and esters of a dimer diol and of a diacid.

The esters of a dimer diol and of a monocarboxylic acid may be obtained from a monocarboxylic acid comprising from 4 to 34 carbon atoms, for example, from 10 to 32 carbon atoms, which acids may be saturated or unsaturated and linear or branched.

Examples of monocarboxylic acids suitable for the present disclosure include, but are not limited to, fatty acids.

The esters of a dimer diol and of a dicarboxylic acid may be obtained from a dimer diacid derived in particular from the dimerization of an unsaturated fatty acid, for example, a C₈ to C₃₄, C₁₂ to C₂₂, or C₁₈ to C₂₀, unsaturated fatty acid.

According to one embodiment, the esters of a dimer diol and of a dicarboxylic acid may be the dimer diacid from which the dimer diol to be esterified also derives.

The dimer diol esters may be obtained from a dimer diol produced by catalytic hydrogenation of a dimer diacid as described above, for example hydrogenated dilinoleic diacid.

Non-limiting examples of dimer diol esters include esters of dilinoleic diacids and of dimer dilinoleyl diols sold by Nippon Fine Chemical under the trade names Lusplan DD-DA5® and DD-DA7®.

silicone oils, such as phenylated silicones, for example Belsil PDM 1000 from Wacker (MM=9000 g/mol),

oils of vegetable origin, such as sesame oil (820.6 g/mol), and

mixtures thereof.

The nonvolatile oils may be present in the composition in an amount ranging from 0.001 to 90% of the total weight of the composition, for example, -from 0.05 to 60%, or from 1 to 35%.

Volatile Oil

The composition of the present disclosure may also comprise at least one volatile oil.

As used herein, the term “volatile oil” is understood to mean an oil (or nonaqueous medium) capable of evaporating on contact with the skin in less than one hour, at ambient temperature and atmospheric pressure. The volatile oil, may be a volatile cosmetic oil which is liquid at ambient temperature and which has, for example, a nonzero vapour pressure, at ambient temperature and atmospheric pressure, such as a vapour pressure ranging from 0.13 Pa to 40 000 Pa (10⁻³ to 300 mmHg), from 1.3 Pa to 13 000 Pa (0.01 to 100 mmHg), or from 1.3 Pa to 1300 Pa (0.1 to 10 mmHg).

In addition, the volatile oil generally has a boiling point, measured at atmospheric pressure, ranging from 150° C. to 260° C., for example, from 170° C. to 250° C.

As used herein, the term “hydrocarbon oil” is understood to mean an oil formed essentially, or composed, of carbon and hydrogen atoms and optionally of oxygen and nitrogen atoms and which does not comprise a silicon or fluorine atom; it may comprise ester, ether, amine, and/or amide groups.

As used herein, the term “silicone oil” is understood to mean an oil comprising at least one silicon atom, for example, comprising Si—O groups.

As used herein, the term “fluorinated oil” is understood to mean an oil comprising at least one fluorine atom.

In one embodiment, the volatile oil may be chosen from silicone oils and hydrocarbon oils.

In another embodiment, the volatile silicone oil may be chosen from silicone oils having a flashpoint ranging from 40° C. to 102° C., for example, greater than 55° C. and less than or equal to 95° C., or ranging from 65° C. to 95° C.

Suitable volatile silicone oils include, but are not limited to, linear or cyclic silicones having a viscosity at ambient temperature of less than 8 cSt and having, for example, from 2 to 7 silicon atoms, these silicones optionally comprising alkyl or alkoxy groups having from 1 to 10 carbon atoms. Examples of such volatile silicone oils include, but are not limited to, octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, and mixtures thereof.

Further examples of suitable volatile silicone oils which can be used in the present disclosure, of the silicones disclosed in the unpublished Application FR 0 304 259.

The volatile hydrocarbon oil which can be used in the present disclosure can be chosen from hydrocarbon oils having a flashpoint ranging from 40° C. to 102° C., for example, from 40° C. to 55° C., or from 40° C. to 50° C.

Further non-limiting examples of volatile hydrocarbon oils include those comprising from 8 to 16 carbon atoms and their mixtures, for example, branched C₈-C₁₆ alkanes, such as C₈-C₁₆ isoalkanes (also known as isoparaffins), isododecane, isodecane, isohexadecane and, for example, the oils sold under the Isopar or Permethyl trade names, branched C₈-C₁₆ esters, such as isohexyl neopentanoate, and their mixtures. In one embodiment, the volatile hydrocarbon oil may be chosen from volatile hydrocarbon oils comprising from 8 to 16 carbon atoms and their mixtures, for example, isododecane, isodecane, and isohexadecane. In another embodiment, the volatile hydrocarbon oil may be isododecane.

The volatile oil may be present in the composition in an amount ranging from 5 to 97.5% of the total weight of the composition, for example, from 10 to 75%, or from 20 and 50% of the total weight of the composition.

In at least one embodiment, the volatile oil may be present in the composition in an amount ranging from 20 to 50% by weight of the composition, for example, from 30 to 40%, or 35% by weight.

Additives

The composition of the present disclosure may also comprise any additional additive conventionally used in the cosmetics field, such as water, antioxidants, preservatives, neutralizing agents, plasticizers, lipophilic gelling agents, nonaqeuous liquid compounds, gelling agents for the aqueous phase, dispersing agents, and cosmetic active principles. These additives, with the exception of water, may be present in the composition in an amount ranging from 0.0005 to 20% of the total weight of the composition, for example, from 0.001 to 10%. The water content of the composition may range from 0 to 70%, for example, from 1 to 50%,or from 1 to 10% by weight, relative to the total weight of the composition

Examples of cosmetic active principles which may be used in the present disclosure include vitamins A, E, C, B₃, and F; provitamins, such as D-panthenol; glycerol; soothing active principles, such as α-bisabolol, aloe vera, allantoin, plant extracts, and essential oils; protecting and/or restructuring agents, such as ceramides; “freshness” active principles, such as menthol and its derivatives; emollients (for example, cocoa butter and dimethicone); moisturizing agents (arginine PCA); antiwrinkle active principles; essential fatty acids; sunscreen agents; and mixtures thereof.

A person skilled in the art will take care to choose the optional additional additives and/or their amounts so that the advantageous properties of the composition according to the present disclosure are not, or not substantially, detrimentally affected by the envisaged addition.

Formulation Forms

The applications of the compositions according to the present disclosure are many fold and relate to all colored or uncolored cosmetic products, for example, lipsticks.

The composition of the present disclosure may be provided in a form chosen from solid, pasty, and liquid compositions, the solid composition being compacted or cast, for example, as a stick or in a dish. In at least one embodiment, the composition may be provided in the solid form, for instance, in the hard form (not flowing under its own weight), and cast or compacted, for example, as a stick or in a dish.

The composition may be provided in the paste, solid, or cream form. It may be in a form chosen from oil-in-water emulsions, water-in-oil emulsions, solid anhydrous gels, soft anhydrous gels, free or compacted powders, and two-phase forms. In at least one embodiment, the compositoin may be provided in the form of a composition with an oily continuous phase which, for example, may be anhydrous; in which case, it may comprise an aqueous phase at a concentration of less than 5%.

The composition according to the present disclosure may be provided in the form of a colored or uncolored composition for caring for the skin, in the form of an antisun or make-up-removing composition, or in the form of a hygiene composition. If it comprises cosmetic active principles, it may then be used as a nontherapeutic treatment or care base for the skin, such as the hands or face, or for the lips (lip balms, which protect the lips from the cold and/or the sun and/or the wind), or a product for the artificial tanning of the skin.

The composition of the present disclosure may also be provided in the form of a colored make-up product for the skin, for example, for the face, such as blushers, face powders, and eyeshadows, a make-up product for the body, such as semipermanent body painting products, a make-up product for the lips, such as lipsticks and lip glosses, optionally exhibiting nontherapeutic treatment or care properties, or a make-up product for the superficial body growths, for example, nail varnishes, mascaras, eyeliners, and products for coloring or caring for the hair.

In one embodiment, the composition according to the present disclosure may be provided in the form of a lipstick or a lip gloss.

The composition of the present disclosure should be physiologically acceptable (for example, cosmetically acceptable), namely nontoxic and capable of being applied to the skin, superficial body growths, and/or lips of human beings.

As used herein, the term “cosmetically acceptable” is understood to mean pleasant with regard to taste, touch, appearance, and/or smell and applicable several days for several months.

The composition according to the present disclosure may be manufactured by known processes generally used in the cosmetics field.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. All amounts, unless otherwise specified, are given as percentages by weight.

### EXAMPLE

Stick of Lipstick:

| Phase | INCI name | Trade name | % |
|---|---|---|---|
| A oil | Octyldodecyl PPG-3 myristyl | Liquiwax PolyEFA | 23 |
| | ether dimer dilinoleate | | |
| | Hydrogenated vegetable oil | Cegesoft HF 52 | 5 |
| C waxes | Wax alcohol | Performacol 550 | 3.25 |
| | | Alcohol | |
| | Polyethylene wax | Polywax 500 | 12 |
| D | Pigment pastes | | 15.25 |
| E | VP/Eicosene copolymer | Antaron V220 | 2 |
| F | Sucrose acetate isobutyrate | Eastman SAIB | 6 |
| | | Special | |
| G gel | Isododecane (and) acrylate | Polymer dispersion* | 9 |
| | copolymer (and) ethylene/ | | |
| | propylene/styrene copolymer | | |
| | Cyclopentasiloxane (and) | Mirasil C-DPDM | 1 |
| | diphenyl dimethicone | | |
| H | Isododecane (and) acrylate | Polymer dispersion* | 23 |
| | copolymer (and) ethylene/ | | |
| | propylene/styrene copolymer | | |
| I | Fragrance | | 0.5 |

Preparation of the Polymer Dispersion

A dispersion of noncrosslinked copolymers of methyl acrylate and of acrylic acid, in a 95/5 ratio, in isododecane was prepared according to the method of Example 1 of European Patent Application No. 0 749 746, with the exception that heptane was replaced with isododecane. A dispersion of poly(methyl acrylate/acrylic acid) particles stabilized at the surface in isododecane by a polystyrene/copoly(ethylene-propylene) sequential diblock copolymer sold under the name of Kraton G1701, having a dry matter content of 25% by weight, was thus obtained.

Procedure:

The pigment paste was prepared with hydrogenated polyisobutene and poly(hydroxystearic acid) in a Discontimill. The milling lasted approximately 30 min.

The gel phase was prepared in a jacketed heating pan under a Rayneri stirrer under cold conditions for 30 min.

The waxes, the pigment paste (phase D), the phase E, and the phase F were weighed out into a jacketed heating pan. The mixture was heated until the waxes had completely melted and were completely homogenized using a deflocculating paddle. The phase A, the phase G, the phase H, and the phase I were then added and the paste was cast at 95° C. in a mould at 40-42° C. and placed in a freezer for 30 min (for 200 g of paste) before being packaged in the pen form.

What is claimed is:

1. A composition comprising
(a) at least one ester of an alkoxylated alcohol and of a carboxylic acid and
(b) at least one noncrystalline film-forming polymer which is solid at ambient temperature, wherein the alkoxylated ester is chosen from compounds of the following formula: 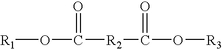
in which:
R₁ has the formula: 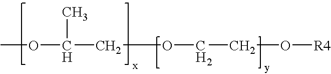
in which:
R₄ is chosen from saturated or unsaturated and substituted or unsubstituted aliphatic units comprising from 4 to 24 carbon atoms; and
x is an integer ranging from 3 to 30; and y is an integer ranging from 3 to 30;
R₂ is chosen from saturated or unsaturated and substituted or unsubstituted aliphatic units comprising from 4 to 40 carbon atoms; and
R₃ is chosen from saturated or unsaturated straight-chain or branched-chain aliphatic units comprising from 4 to 32 carbon atoms.

2. The composition of claim 1, wherein R₄ is a saturated aliphatic unit comprising from12 to 20 carbon atoms.

3. The composition of claim 1, wherein R₂ is a saturated aliphatic unit comprising from 4 to 40 carbon atoms.

4. The composition of claim 1, wherein x and y are, independently of one another, equal to 3 or 4.

5. The composition of claim 1, wherein R₃ is a branched-chain saturated aliphatic unit comprising from 12 to 20 carbon atoms.

6. The composition of claim 4, wherein R₃ is chosen from octyidodecyl, isostearyl, and stearyl.

7. The composition of claim 1, wherein the at least one alkoxylated ester is Octyldodecyl PPG-3 Myristyl Ether Dimer Dilinoleate.

8. The composition of claim 1, wherein the at least one alkoxylated ester is Isostearyl PPG-4 Butyloctyl Ether Dimer Dilinoleate.

9. The composition of claim 1, wherein the polymer is a film-forming polymer which, when present in the composition in a sufficient amount, allows said composition to be capable of forming a deposited layer having a hold of greater than or equal to 30%.

10. The composition of claim 1, wherein the at least one film-forming polymer is chosen from silicone gums.

11. The composition of claim 1, wherein the at least one film-forming polymer is chosen from silicone resins.

12. The composition of claim 1, wherein the at least one film-forming polymer is chosen from block acrylic copolymers, radical acrylic copolymers, acrylic polymers grafted with a silicone macromonomer, and mixtures thereof.

13. The composition of claim 1, wherein the at least one film-forming polymer is polyisoprene.

14. The composition of claim 1, wherein the at least one film-forming polymer is a polyamide/polysiloxane copolymer.

15. The composition of claim 14, wherein the composition is capable of forming a deposited layer having a hold index of greater than or equal to 40%.

16. The composition according to claim 15, wherein the composition is capable of forming a deposited layer having a hold index of greater than or equal to 50%.

17. The composition of claim 16, wherein the composition is capable of forming a deposited layer having a hold index of greater than 60%

18. The composition of claim 17, wherein the composition is capable of forming a deposited layer having a hold index of greater than or equal to 65%.

19. The composition of claim 1, further comprising at least one coloring material chosen from dyes, pigments, pearlescent agents, and glitter.

20. The composition of claim 1, further comprising at least one fatty substance chosen from waxes, pasty fatty substances, oils, and mixtures thereof.

21. The composition of claim 1, further comprising at least one cosmetic ingredient chosen from vitamins, thickeners, gelling agents, trace elements, softeners, sequestering agents, fragrances, basifying agents, acidifying agents, preservatives, sunscreen agents, surfactants, antioxidants, fibers, agents for combating hair loss, agents for caring for the eyelashes, antidandruff agents, propellants, and mixtures thereof.

22. The cosmetic composition of claim 1, wherein the composition is provided in a form chosen from suspensions, dispersions, solutions, gels, emulsions, creams, pastes, foams, dispersions of vesicles, two-phase lotions, multiphase lotions, sprays, powders, sticks, and cast solids.

23. The cosmetic composition of claim 22, wherein the emulsions are chosen from oil-in-water (O/W), water-in-oil (W/O), and multiple emulsions.

24. The composition of claim 23, wherein the multiple emulsions are chosen from water-in-oil-in-water (W/O/W), polyol-in-oil-in water (polylol/O/W), and oil-in-water-in-oil (O/W/O) emulsions.

25. The cosmetic composition of claim 22, wherein the pastes are chosen from soft pastes and anhydrous pastes.

26. The cosmetic composition of claim 22, wherein the dispersions of vesicles are chosen from dispersions of ionic lipid vesiclse and dispersions of nonionic lipid vesicles.

27. The cosmetic composition of claim 1, wherein it is provided in the anhydrous form.

28. The cosmetic composition of claim 1, wherein it is a composition for making up or caring for keratinous substances.

29. The composition of claim 1, wherein it is a product for making up the lips.

30. A composition comprising
(a) at least one ester of an alkoxylated alcohol and of a carboxylic acid and
(b) at least one noncrystalline film-forming polymer chosen from vinyl polymers and copolymers, urethane polymers and copolymers, polyester polymers and copolymers, polyamide polymers and copolymers, polyurea polymers and copolymers, cellulose polymers and copolymers, and silicone polymers and copolymers.

31. The composition of claim 30, wherein the vinyl polymers and copolymers are chosen from acrylic and ethylenic polymers and copolymers.

32. The composition of claim 30, wherein the polyamide polymers and copolymers are chosen from silicone polyamides.

33. The composition of claim 30, wherein the cellulose polymers and copolymers are chosen from nitrocellulose.

34. A method for making up and/or caring for the skin, lips, and/or superficial body growths comprising applying, to the skin, lips, and/or superficial body growths, at least one cosmetic composition, wherein the cosmetic composition comprises
(a) at least one ester of an alkoxylated alcohol and of a carboxylic acid and
(b) at least one noncrystalline film-forming polymer which is solid at ambient temperature, wherein the alkoxylated ester is chosen from compounds of the following formula: 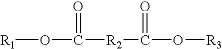
in which:
R₁ has the formula: 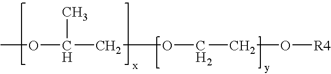
in which:
R₄ is chosen from saturated or unsaturated and substituted or unsubstituted aliphatic units comprising from 4 to 24 carbon atoms; and
x is an integer ranging from 3 to 30; and y is an integer ranging from 3 to 30;
R₂ is chosen from saturated or unsaturated and substituted or unsubstituted aliphatic units comprising from 4 to 40 carbon atoms; and
R₃ is chosen from saturated or unsaturated straight-chain or branched-chain aliphatic units comprising from 4 to 32 carbon atoms.
